SEQUENCE LISTING (8 sequences)

SEQ ID NO: 1 - protein, 16 aa, Artificial Sequence
  Artificial Sequence Note = Synthetic Construct
PCFXXXXXDPXXXXXR

SEQ ID NO: 2 - protein, 12 aa, Artificial Sequence
  Artificial Sequence Note = Synthetic Construct
PAGSLDPAGSLR

SEQ ID NO: 3 - protein, 14 aa, Artificial Sequence
  Artificial Sequence Note = Synthetic Construct
PAGSMLDPAGSMLR

SEQ ID NO: 4 - protein, 12 aa, Artificial Sequence
  Artificial Sequence Note = Synthetic Construct
PAGSLADPGSLR

SEQ ID NO: 5 - protein, 12 aa, Artificial Sequence
  Artificial Sequence Note = Synthetic Construct
PALSLADPGSGR

SEQ ID NO: 6 - protein, 12 aa, Artificial Sequence
  Artificial Sequence Note = Synthetic Construct
PALSLGDPASGR

SEQ ID NO: 7 - protein, 12 aa, Artificial Sequence
  Artificial Sequence Note = Synthetic Construct
PAGSDPLAGSLR

SEQ ID NO: 8 - protein, 12 aa, Artificial Sequence
  Artificial Sequence Note = Synthetic Construct
PADPGSLAGSLR

(19) United States
(12) Patent Application Publication
(10) Pub. No.: US 2003/0124595 A1
(43) Pub. Date: Jul. 3, 2003

(54) Sensitive coded detection systems

(76) Inventor: Paul M. Lizardi, Wallingford, CT (US)

Correspondence Address: NEEDLE & ROSENBERG P C, 127 PEACHTREE STREET N E, ATLANTA, GA, 30303-1811, US

(21) Appl. No.: 10/289,118

(22) Filed: Nov. 5, 2002

Related U.S. Application Data
(60) Provisional application No. 60/332,982, filed on Nov. 6, 2001.

(51) Int. Cl. C12Q 1/68

(52) U.S. Cl. 435/6

(57) ABSTRACT

Disclosed are compositions and methods for sensitive multiplex detection of analytes. The disclosed compositions, referred to as detectors, accomplish this detection by associating specific binding molecules—which interact with desired targets—with block groups in a carrier. The block groups are made up of blocks which, through the combination of different blocks, constitute a code for a given detector. The blocks are detectable and each detector is distinguishable from other detectors by its block group. The coding of the block groups greatly increasing the number of distinguishable detectors from a relatively small number of blocks. The detection burden remains low even with such a large number of block groups because only the blocks need be distinguished from each other during detection. Numerous block molecules of each type making up the block group can be present in the carrier to effectively amplify the signal generated from targets.

### CROSS-REFERENCE TO RELATED APPLICATIONS

This application claims benefit of U.S. Provisional Application No. 60/332,982, filed Nov. 6, 2001. Application Ser. No. 60/332,982, filed Nov. 6, 2001, application Ser. No. 09/850,539, filed May 7, 2001, and application Ser. No. 09/929,266, filed Aug. 13, 2001, are hereby incorporated herein by reference in their entirety.

### FIELD OF THE INVENTION

The present invention is generally in the field of detection of molecules, and specifically in the field of detection of multiple different molecules in a single assay.

### BACKGROUND OF THE INVENTION

The analysis of proteins in histological sections and other cytological preparations is routinely performed using the techniques of histochemistry, immunohistochemistry, or immunofluorescence. By performing immunofluorescence with antibodies labeled with different colors, it has been possible to detect simultaneously 2, 3, or even 4 different antigens present in cellular material. In the future, time-resolved fluorescence may permit the extension of immunofluorescence methods to the detection of 6 to 12 different antibodies simultaneously. Likewise, RNA detection by fluorescence in situ hybridization permits the detection of 2 to 4 different RNAs in cellular material, and it may also be extended to permit the detection of 6 to 12 different RNAs by time-resolved fluorescence.

There is a need for a sensitive method that will permit the cytological detection of larger numbers of proteins or RNAs simultaneously. Theoretically, the simultaneous measurement of the concentration of 20 to 50 different protein (or RNA) species should be highly informative as to the specific status of dynamic cellular processes in normal development, in stages of disease, in response to drug treatment or gene therapy, or as a result of environmental exposure or other deliberate or inadvertent interventions.

The study of cells by measuring the identity and concentration of a relatively large number of proteins simultaneously (referred to as proteomics) is currently a very time-consuming task. Two-dimensional (2D) gel electrophoresis is a useful tool for studying the expression of multiple proteins, but this technique is not readily adaptable to in-situ cell analysis. Typically, many thousands of cells are required to perform a single 2D gel analysis. In order to identify different protein expression profiles in heterogeneous tissue samples, one would need the capability to analyze the proteins expressed in a small number of cells. This capability is most relevant in the analysis of histological or cytological specimens that may harbor dysplastic or pre-malignant cells. Such cells, which may precede the development of cancer, need to be identified when present as small foci of 10 to 50 cells, before they have a chance to give rise to tumors. Unfortunately, the amount of protein obtained from 10 to 50 cells is insufficient for 2D gel analysis, and is problematic even with the use of radioisotopes to label the protein.

Mass spectroscopy is another powerful technique for protein analysis. However, the direct analysis of proteins present in samples containing small numbers of cells is not possible with prior mass spectroscopy technology, due to insufficient sensitivity. A minimum of 10,000 cells is required for mass spectroscopic analysis of tissue samples using prior technology.

Current methods for the analysis of microarray hybridization experiments rely on the use of a two-color signal readout system. For example, Schena M, Shalon D, Davis R W, Brown P O (1995) Quantitative monitoring of gene expression patterns with a complementary DNA microarray. Science 270:467-70, describe an experiment where cDNA prepared from one tissue is labeled with the dye cy3, while cDNA from another tissue is labeled with the dye cy5. After the labeling reactions are performed, the two labeled DNAs are mixed, and hybridized by contacting with the surface of a glass slide containing a cDNA microarray on its surface. At the end of the hybridization reaction, the microarray surface is washed to remove unhybridized material, and the glass slide is scanned in a confocal scanning instrument designed to record separately the cy3 and the cy5 fluorescence intensity, which is saved as two different computer files. Computer software is then used to calculate the fluorescence ratio of cy3 to cy5 at each of the specific dot-addresses on the DNA microarray. This-experimental design works very well for performing comparisons of mRNA expression ratios between two samples.

Gygi S P, Rist B, Gerber S A, Turecek F, Gelb M H, Aebersold R (1999) Quantitative analysis of complex protein mixtures using isotope-coded affinity tags. Nature Biotechnology 17:994-999, have described an approach for the accurate quantification and concurrent sequence identification of the individual proteins within complex mixtures of biological origin. The method is based on a class of new chemical reagents termed isotope-coded affinity tags (ICATs), and mass spectrometry. These authors extracted proteins from two different experimental states of an organism, and labeled each of the two preparations of total protein with two different thiol-reactive ICAT tags of different mass. The two labeled protein preparations were mixed, separated by liquid chromatography, and detected on line by mass spectrometry. For each individual protein peak, mass spectrometry permitted protein identification, as well as measurement of the ratio of the amounts of the two proteins.

### BRIEF SUMMARY OF THE INVENTION

Disclosed are compositions and methods for sensitive multiplex detection of analytes. The system is designed for the simultaneous detection of dozens or even hundreds of analytes. The analytes can be detected in any context. For example, the analytes may be present on the surface of cells in suspension, on the surface of cytology smears, on the surface of histological sections, on the surface of DNA microarrays, on the surface of protein microarrays, on the surface of beads, or any other situation where complex samples need to be studied. The disclosed compositions, referred to as detectors, accomplish this detection by associating specific binding molecules—which interact with desired targets—with block groups in a carrier. The block groups are made up of blocks which, through the combination of different blocks, constitute a code for a given detector. The blocks are detectable and each detector is distinguishable from other detectors by its block group. The coding of the block groups greatly increasing the number of distinguishable detectors from a relatively small number of blocks. For example, the multiplexing possibilities from twenty blocks combined in block groups of five different blocks each amount to 15,504 distinguishable combinations. The detection burden remains low even with such a large number of block groups because only the blocks need be distinguished from each other during detection. Numerous block molecules of each type making up the block group can be present in the carrier to effectively amplify the signal generated from targets.

It is an object of the present invention to provide a composition that permits the indirect detection of a large number of different analytes in a single sample or group of samples.

It is another object of the present invention to provide a composition that permits the indirect detection of a large number of different proteins in a single sample or group of samples.

### BRIEF DESCRIPTION OF THE DRAWINGS

FIG. 1 is a diagram illustrating the components of the disclosed detectors. Detector 101 is composed of carrier 102 to which specific binding molecule 103 and block group 104 are attached. Block group 104 is composed of blocks 105.

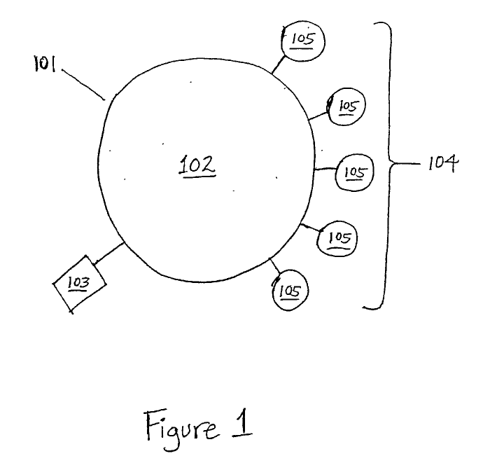

### DETAILED DESCRIPTION OF THE INVENTION

Methods currently in use for the detection of protein, DNA, or RNA in biological material are limited to the use of just a few analytes as targets. There is a need for methods that enable the simultaneous detection of a large number of analytes. Certain microarray methods solve this multiplexing requirement by distributing samples so they are physically separated at different addresses on a surface. However, there are certain types of analysis where the analytes in a sample of interest are all present in a single address or location, and can not be separated physically. For example, one may desire to detect 40 different proteins simultaneously, with fairly accurate cellular localization, on the surface of a tissue section (fairly accurate means that the proteins are detected in the vicinity of a group of 20 cells or less. This type of information may be useful, for example, to decide if a certain group of cells has undergone neoplastic transformation.

Different embodiments of the present compositions and method allow the detection of protein, RNA, DNA, carbohydrate, or any other analyte of interest, based on the use of specific recognition moieties, referred to as specific binding molecules, for each of these analytes. For example, a useful recognition moiety for a protein analyte is an antibody specific for an epitope present in that protein, while a useful recognition moiety for a nucleic acid analyte is a complementary nucleic acid probe.

The disclosed compositions, referred to herein as detectors, are based on the use of carriers comprising a set of arbitrary molecular tags that have been optimized to facilitate a subsequent detection. The molecular tags are referred to as blocks and the set of blocks is referred to as a block group. The carriers are linked, preferably by covalent coupling, to specific recognition molecules. The specific recognition molecules are referred to as specific binding molecules. The detectors, by virtue of the directly or indirectly linked recognition molecules, may be used as reporters in bioassays. The blocks can be optimized by their chemical composition, so that they may be efficiently separated by, for example, mass spectrometry. Blocks to be separated by mass spectrometry will differ in molecular weight, preferably by well resolved mass differences that allow for reliable separation. For separation by mass spectrometry, the carriers can be loaded with reporter signals where differences between the mass-to-charge ratio of altered forms of the reporter signals can be used to distinguish and detect the carriers.

Although specific terms are used herein to name particular components of the disclosed compositions and methods, such terms are not intended to limit the scope and nature of the components. Rather, the definitions, descriptions, illustrations, examples, and other references herein to the components are intended to define the scope and nature of the components.

### Materials

A. Detectors

Detectors are associations of one or more specific binding molecules, a carrier, and a block group. Block groups are sets of blocks. Detectors are used in the disclosed method to associate a block group with a target molecule. The carrier can be any molecule or structure that facilitates association of block groups with a specific binding molecule. Examples include beads, including, for example, microbeads and nanobeads; liposomes; particles, including, for example, microparticles and nanoparticles; and polymers, including, for example, branched polymer structures. The are three useful types of detectors: liposome detectors, dendrimer detectors, and bead detectors. Carriers can be made from a variety of substances including acrylamide, cellulose, nitrocellulose, glass, polystyrene, polyethylene vinyl acetate, polypropylene, polymethacrylate, polyethylene, polyethylene oxide, glass, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, polyamino acids, controlled release polymers, gels, insoluble polymers, bioerodible polymers, monolayers, bilayers, vesicles, liposomes, membranes, resins, matrices, fibers, separation media, chromatography supports, hydrogels, polymers, plastics, glass, mica, gold, beads, microbeads, nanobeads, microspheres, nanospheres, particles, microparticles, nanoparticles, silicon, gallium arsenide, organic and inorganic metals, semiconductors, insulators, microstructures, and nanostructures.

Carriers can have any useful form, including beads, bottles, dishes, disks, compact disks, fibers, optical fibers, woven fibers, shaped polymers, particles, and microminiaturized, micrometer-scale, nanometer-scale and supramolecular forms of beads, particles, probes, tips, bars, pegs, plugs, rods, sleeves, wires, filaments, tubes, ropes, tentacles, tethers, chains, capillaries, vessels, walls, edges, corners, seals, channels, lips, lattices, trellises, grids, arrays, knobs, steps, arms, teeth, cords, surfaces, layers, films, polymers, and membranes.

The disclosed detectors combine carriers and arbitrary block groups. By combining detectors, associated with arbitrary block groups, with methods capable of separating a multiplicity of blocks, it becomes possible to perform highly multiplexed assays.

Although the components of detectors are referred to herein in the singular, detectors can include a plurality of any of the components. For example, a detector referred to as containing a block group can have multiple copies of the same block group (that is, multiple copies of the blocks making up the block group). However, unless otherwise indicated, in the context of a detector, reference to a specific binding molecule in the singular indicates a single molecule.

Beads are a useful form of carrier. Beads can be made from any suitable substance, preferably from polymer(s). For example, beads can be made from acrylamide, cellulose, nitrocellulose, glass, polystyrene, polyethylene vinyl acetate, polypropylene, polymethacrylate, polyethylene, polyethylene oxide, glass, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, polyamino acids, controlled release polymers, insoluble polymers, and bioerodible polymers. Beads can be of any desired size. For example, beads can be from about 0.2 microns to about 250 microns in diameter, from about I micron to about 250 microns in diameter, from about 2 microns to about 250 microns in diameter, from about 5 microns to about 250 microns in diameter, from about 10 microns to about 250 microns in diameter, from about 20 microns to about 250 microns in diameter, from about 30 microns to about 250 microns in diameter, from about 0.2 microns to about 200 microns in diameter, from about 0.2 microns to about 150 microns in diameter, from about 0.2 microns to about 100 microns in diameter, from about 0.2 microns to about 80 microns in diameter, from about 0.2 microns to about 50 microns in diameter, from about 0.2 microns to about 40 microns in diameter, from about 0.2 microns to about 30 microns in diameter, from about 0.2 microns to about 20 microns in diameter, from about 0.2 microns to about 15 microns in diameter, from about 0.2 microns to about 10 microns in diameter, from about 0.2 microns to about 5 microns in diameter, from about 0.2 microns to about 2 microns in diameter, from about 0.2 microns to about 1 micron in diameter, from about 1 micron to about 200 microns in diameter, from about 1 micron to about 150 microns in diameter, from about 1 micron to about 100 microns in diameter, from about 1 micron to about 80 microns in diameter, from about 1 micron to about 50 microns in diameter, from about 1 micron to about 40 microns in diameter, from about I micron to about 30 microns in diameter, from about 1 micron to about 20 microns in diameter, from about 1 micron to about 15 microns in diameter, from about 1 micron to about 10 microns in diameter, from about 1 micron to about 5 microns in diameter, from about 1 micron to about 2 microns in diameter, from about 2 microns to about 200 microns in diameter, from about 2 microns to about 150 microns in diameter, from about 2 microns to about 100 microns in diameter, from about 2 microns to about 80 microns in diameter, from about 2 microns to about 50 microns in diameter, from about 2 microns to about 40 microns in diameter, from about 2 microns to about 30 microns in diameter, from about 2 microns to about 20 microns in diameter, from about 2 microns to about 15 microns in diameter, from about 2 microns to about 10 microns in diameter, from about 2 microns to about 5 microns in diameter, from about 3 microns to about 200 microns in diameter, from about 3 microns to about 150 microns in diameter, from about 3 microns to about 100 microns in diameter, from about 3 microns to about 80 microns in diameter, from about 3 microns to about 50 microns in diameter, from about 3 microns to about 40 microns in diameter, from about 3 microns to about 30 microns in diameter, from about 3 microns to about 20 microns in diameter, from about 3 microns to about 15 microns in diameter, from about 3 microns to about 10 microns in diameter, from about 3 microns to about 5 microns in diameter, from about 5 microns to about 200 microns in diameter, from about 5 microns to about 150 microns in diameter, from about 5 microns to about 100 microns in diameter, from about 5 microns to about 80 microns in diameter, from about 5 microns to about 50 microns in diameter, from about 5 microns to about 40 microns in diameter, from about 5 microns to about 30 microns in diameter, from about 5 microns to about 20 microns in diameter, from about 5 microns to about 15 microns in diameter, from about 5 microns to about 10 microns in diameter, from about 10 microns to about 200 microns in diameter, from about 10 microns to about 150 microns in diameter, from about 10 microns to about 100 microns in diameter, from about 10 microns to about 80 microns in diameter, from about 10 microns to about 50 microns in diameter, from about 10 microns to about 40 microns in diameter, from about 10 microns to about 30 microns in diameter, from about 10 microns to about 20 microns in diameter, from about 10 microns to about 15 microns in diameter, from about 20 microns to about 200 microns in diameter, from about 20 microns to about 150 microns in diameter, from about 20 microns to about 100 microns in diameter, from about 20 microns to about 80 microns in diameter, from about 20 microns to about 50 microns in diameter, from about 20 microns to about 40 microns in diameter, and from about 20 microns to about 30 microns in diameter.

Although specific bead sizes and specific endpoints for ranges of the bead size are recited, each and every specific bead size and each and every specific endpoint of ranges of bead size are specifically contemplated, although not explicitly listed, and each and every specific bead size and each and every specific endpoint of ranges of bead size are hereby specifically described. Beads to be used as carriers in the same set of detectors or in the same assay or other group or use can have the same or similar size and dimensions. However, this is not required and beads of varying size and dimension can be used. By same size is meant a size within about 5% of a reference size (giving a possible spread of about 10%). By same dimensions is meant dimensions within about 5% of a reference size (giving a possible spread of about 10%). By similar size is meant a size within about 30% of a reference size (giving a possible spread of about 60%). By similar dimensions is meant dimensions within about 30% of a reference size (giving a possible spread of about 60%). Although preferred, beads need not be spherical. In this regard, reference to diameter of beads is not intended to imply that the beads are spherical. Rather, as used herein, the “diameter” of a bead refers to the length of the longest dimension.

Liposomes are artificial structures primarily composed of phospholipid bilayers. Cholesterol and fatty acids may also be included in the bilayer construction. Liposomes may be loaded with fluorescent tags, and coated on the outer surface with specific recognition molecules (Truneh, A., Machy, P. and Horan, P. K., 1987, Antibody-bearing liposomes as multicolor immunofluorescent markers for flow cytometry and imaging. J. Immunol. Methods 100:59-71). However, the use of fluorescent liposomes in bioassays has been limited by the constraints of detection methods for fluorescent tags. Fluorescence-activated cell sorters typically have two or three different excitation-emission wavelengths, and microscopes typically have three or four excitation-emission filters. In the disclosed liposome detectors, liposomes serve as carriers for arbitrary block groups. By combining liposome detectors, loaded with arbitrary block groups, with methods capable of separating a multiplicity of blocks, it becomes possible to perfonn highly multiplexed assays.

Liposomes, such as unilamellar vesicles, are made using established procedures that can result in the loading of the interior compartment with a very large number (several thousand) of block molecules, where the chemical nature of these molecules is well suited for detection by a preselected detection method.

Each specific type of liposome detector is associated with a specific binding molecule. The association may be direct or indirect. An example of a direct association is a liposome containing covalently bound antibodies on the surface of the phospholipid bilayer. An example of indirect association is a liposome containing covalently bound nucleic acid of arbitrary sequence on its surface. These oligonucleotides are designed to recognize, by base complementarity, specific oligonucleotides coupled to particular specific binding molecules. In this fashion, the liposome detector becomes a generic reagent, which may be associated indirectly with any desired binding molecule.

The synthesis of dendrimers that may be used as polylabeled DNA probes has been described (Schchepinov, M. S., Udalova, I. A., Bridgman, A. J., Southern, E. M., 1997, Nucleic Acids Res. 25:4447-4454). Dendrimers may be associated with block groups to form dendrimer detectors.

B. Block Groups

A block group is a set of blocks that can be associated with a carrier in a detector. Block groups can be used to distinguish detectors by using different block groups in different detectors. Block groups are particularly useful when used in sets where each different block group in a set can be distinguished from other block groups in the set. Such sets of block groups are useful for use in sets of detectors where each detector in the set can be distinguished from other detectors in the set. This can be accomplished, for example, by using a different block group (from the set of block groups, for example) for each detector.

Sets of block groups can be made up of block groups having any desired or useful relationship. Generally, block groups in a set can have particular relationships to each other. For example, the members of a set of block groups can be related such that each different block group in a set can be distinguished from other block groups in the set. This can be accomplished, for example, by using block groups that each have a different composition of blocks. By composition of blocks is meant the identity, amount, or identity and amount of blocks. Composition of blocks based only on identity is referred to as the identity composition of blocks. Composition of blocks based only on amount is referred to as the amount composition of blocks. Composition of blocks based on both identity and amount is referred to as the overall composition of blocks. The identify composition of a block group refers to the identity of the blocks in the block group. The amount composition of a block group refers to the amount of the blocks in the block group. The overall composition of a block group refers to the identity and amount of the blocks in the block group.

By identity of block is meant a particular block, but not a particular block molecule. Thus, a block molecule composed of the peptide AGSLADPGSLR (SEQ ID NO:4) has the same identity as a different block molecule composed of the peptide AGSLADPGSLR (SEQ ID NO:4) but a different identity from a block molecule composed of the peptide ALSLADPGSGR (SEQ ID NO:5).

By amount of block is meant the number of molecules of a block (where the number of molecules can be referred to by any appropriate or convenient measure, such as by mass or by mole, including by submolar units). For practical purposes, blocks in a block group can be composed of multiple block molecules having the same identity (that is, in a detector, each block in the block group can be represented by multiple physical molecules). However, for convenience such collections of multiple block molecules having the same identity will be referred to in the singular as a block. The amount of a block used in a block group can be significant, for example, in establishing a ratio of the amount the different blocks in a block group. For example, a block group may be composed of three blocks of different identity with one of the blocks present in twice the amount of the other two blocks. Such differences in the amount or ratio of blocks are detected in some forms of the disclosed compositions and methods. However, differences in the amount of blocks present in a block group need not be given effect. For example, in some forms of the disclosed compositions and methods the identity, and not the amount or ratio, of blocks in a block group is detected and analyzed. In other forms of the disclosed compositions and methods, both the identity and amount or ratio of blocks in a block group can be detected and analyzed.

The amount composition of blocks in block groups can be the same or different. That is, there can be substantially the same amount of each of the different blocks in a block group or there can be different amounts of one or more of the different blocks in a block group. A block group where each of the blocks is present in substantially the same amount is referred to as having a level amount composition. By substantially the same amount is meant a difference in amount of about 10% or less. A block group where one or more of the blocks is present in a different amount from other blocks in the block group is referred to as having an unbalanced amount composition. By different amount is meant a difference in amount of about 20% or more. A set of block groups where the block groups have level amount composition are referred to as level amount composition block group sets. A set of block groups where one or more of the block groups have unbalanced amount composition are referred to as unbalanced amount composition block group sets.

1. Specific-Number Block Group Sets

The identity composition of blocks in block groups can be varied in a variety of ways. In particular, a set of block groups can be characterized by the relationship of different identity compositions of blocks for the different block groups in the set. For example, block groups in a set of block groups can be composed the same number of different block (that is, blocks of different identity). This is referred to as a specific-number block group set. To illustrate, there can be a set of block groups each composed of three different blocks. The identity composition of each block group (that is, the identity of the three blocks making up that block group) can be different for each block group in the set. To further illustrate, consider a set of block groups where each block group is composed of three different blocks chosen from a set of ten blocks (identified in this illustration as A, B, C, D, E, F, G, H, I, and J). The identity composition of the block groups in the set could be:

ABC, ABD, ABE, ABF, ABG, ABH, ABI, ABJ, ACD, ACE, . . . AGJ, AHI, AHJ, AIJ, BCD, BCE, BCF, BCG, BCH, BCI, BCJ, BDE, BDF, . . . EIJ, FGH, FGI, FGJ, FHI, FHJ, FIJ, GHI, GHJ, GIJ, HIJ.

Note that all of the block groups are composed of exactly three different blocks, excluding combinations such as AAB, ADD, AAA. The excluded combinations have identify compositions of only one or two different blocks, which is outside the scope of this set of block groups. Note also that order does not matter. A block group having the identity composition of ABC is the same as a block group having the identity composition ACB. It should be understood that this illustration involves a set of block groups that includes all of the possible identity compositions of blocks meeting the criteria of the block group set. Block group sets can also be composed of less than all of the possible identity compositions of blocks meeting the criteria of the block group set. A specific-number block group set having less than all of the possible identity compositions of blocks meeting the criteria of the block group set is still referred to as a specific-number block group set.

The amount composition of blocks in a specific-number block group set can be the same or different. That is, there can be substantially the same amount of each of the different blocks in a block group or there can be different amounts of one or more of the different blocks in a block group. Thus, a specific-number block group set can be either a level amount composition block group set or an unbalanced amount composition block group set. To illustrate, using the specific-number block group set described above, an unbalanced amount composition specific-number block group set could include block groups such as A2BC, A2BD, A2BE, A2BF, A2BG, A2BH, A2BI, A2BJ, ACD, ACE, . . . AGJ, AHI, AHJ, AIJ, 2BCD, 2BCE, 2BCF, 2BCG, 2BCH, 2BCI, 2BCJ, 2BDE, 2BDF, . . . EIJ, FGH, FGI, FGJ, FHI, FHJ, FIJ, GHI, GHJ, GIJ, and HIJ, where the number in front of a block refers to the relative amount of that block. In this illustration, block B is present in twice the amount of the other blocks.

2. Variable-Number Block Group Sets

Block groups in a set of block groups also can be composed the different numbers of different blocks. This is referred to as a variable-number block group set. Variable-number block group sets can have a range of the number of blocks per block group. A variable-number block group set can have, for example, block groups with two blocks and block groups with three blocks; block groups with three blocks, block groups with four blocks, and block groups with five blocks; block groups with one block, block groups with two blocks, and block groups with three blocks; or block groups with two blocks, block groups with four blocks, and block groups with five blocks. These are just examples; variable-number block group sets can have block groups encompassing a wide range of numbers of blocks per block group. To illustrate, there can be a set of block groups with some of the block groups composed of two different blocks and other block groups composed of three different blocks. The identity composition of each block group (that is, the identity of the two or three blocks making up that block group) can be different for each block group in the set. To further illustrate, consider a set of block groups where each block group is composed of two or three different blocks chosen from a set of ten blocks (identified in this illustration as A, B, C, D, E, F, G, H, I, and J). The identity composition of the block groups in the set could be:

AB, AC, AD, AE, AF, AG, AH, Al, AJ, BC, BD, BE, . . . GH, GI, GJ, HI, HJ, IJ, ABC, ABD, ABE, ABF, ABG, ABH, ABI, ABJ, ACD, ACE . . . AGJ, AHI, AHJ, AIJ, BCD, BCE, BCF, BCG, BCH, BCI, BCJ, BDE, BDF, . . . EIJ, FGH, FGI, FGJ, FHI, FHJ, FIJ, GHI, GHJ, GIJ, HIJ.

Note that all of the block groups are composed of exactly two or exactly three different blocks, excluding combinations such as BB. The excluded combinations have identify compositions of only one block, which is outside the scope of this set of block groups. Note also that order does not matter. A block group having the identity composition of ABC is the same as a block group having the identity composition ACB. It should be understood that this illustration involves a set of block groups that includes all of the possible identity compositions of blocks meeting the criteria of the block group set. Block group sets can also be composed of less than all of the possible identity compositions of blocks meeting the criteria of the block group set. A variable-number block group set having less than all of the possible identity compositions of blocks meeting the criteria of the block group set is still referred to as a variable-number block group set. However, a “variable-number” block group set that excludes all block groups except those block groups having the same number of blocks (for example, three blocks) would be a specific-number block group set.

The amount composition of blocks in a variable-number block group set can be the same or different. That is, there can be substantially the same amount of each of the different blocks in a block group or there can be different amounts of one or more of the different blocks in a block group. Thus, a variable-number block group set can be either a level amount composition block group set or an unbalanced amount composition block group set. To illustrate, using the variable-number block group set described above, an unbalanced amount composition variable-number block group set could include block groups such as A2B, AC, AD, AE, AF, AG, AH, Al, AJ, 2BC, 2BD, 2BE, . . . GH, GI, GJ, HI, HJ, IJ, A2BC, A2BD, A2BE, A2BF, A2BG, A2BH, A2BI, A2BJ, ACD, ACE, . . . AGJ, AHI, AHJ, AIJ, 2BCD, 2BCE, 2BCF, 2BCG, 2BCH, 2BCI, 2BCJ, 2BDE, 2BDF, . . . EIJ, FGH, FGI, FGJ, FHI, FHJ, FIJ, GHI, GHJ, GIJ, and HIJ, where the number in front of a block refers to the relative amount of that block. In this illustration, block B is present in twice the amount of the other blocks.

Another form of variable-number block group set involves block groups where only one of the block groups in the set has any given combination or subcombination of blocks. Thus, in such a block group set, if a two block block group has an identity composition of AB, no other block group should include the combination AB. For example, a block group of identity composition ABC would not be in such a set, but block groups of block group compositions ACD, BCD, AC, and BC could be in the set. Using the block group set described above, a block group set with no combination repeats that includes AB and IJ as the only two block block groups could include ACD, ACE, ACF, ACG, ACH, ACI, ACJ, ADE, ADF, . . . AGJ, AHI, AHJ, BCD, BCE, BCF, BCG, BCH, BCI, BCJ, BDE, BDF, . . . EGH, EGI, EGJ, EHI, EHJ, FGH, FGI, FGJ, FHI, FHJ, GHI, and GHJ, but would not include ABC, ABD, ABE, ABF, ABG, ABH, ABI, ABJ, AIJ, BIJ, CIJ, DIJ, EIJ, FIJ, GIJ, or HIJ. Such a no combination repeat variable-number block group set can be useful for increasing the distinction between different block groups in the set.

3. Variable-Amount Block Group Sets

The amount composition of blocks in block groups can be varied in a variety of ways. In particular, a set of block groups can be characterized by the relationship of different amount compositions of blocks for the different block groups in the set. For example, block groups in a set of block groups can be composed of different amounts of blocks. This is referred to as a variable-anmount block group set. Such sets have unbalanced amount composition. To illustrate, there can be a set of block groups each composed of three blocks in different amounts. The amount composition of each block group (that is, the amount of each of the three blocks making up that block group) can be different for each block group in the set (the identity composition can also differ between block groups). To further illustrate, consider a set of block groups where each block group is composed of three blocks chosen from a set of five blocks (identified in this illustration as A, B, C, D, and E) in three different amounts. The overall composition of the block groups in the set could be:

ABC, ABD, ABE, BCD, BCE, CDE, 2ABC, 2ABD, 2ABE, 2BCD, 2BCE, 2CDE, 3ABC, 3ABD, 3ABE, 3BCD, 3BCE, 3CDE, 4ABC, 4ABD, 4ABE, 4BCD, 4BCE, 4CDE, A2BC, A2BD, A2BE, B2CD, B2CE, C2DE, A3BC, A3BD, A3BE, B3CD, B3CE, C3DE, A4BC, A4BD, A4BE, B4CD, B4CE, C4DE, AB2C, AB2D, AB2E, BC2D, BC2E, CD2E, AB3C, AB3D, AB3E, BC3D, BC3E, CD3E, AB4C, AB4D, AB4E, BC4D, BC4E, CD4E,

2A2BC, . . . 2C2DE, 2A3BC, . . . 2C3DE, 2A4BC, . . . 2C4DE, 3A2BC, . . . 3C2DE, 3A3BC, . . . 3C3DE, 3A4BC, . . . 3C4DE, 4A2BC, . . . 4C2DE, 4A3BC, . . . 4C3DE, 4A4BC, . . . 4C4DE, 2AB2C, . . . 2CD2E, 2AB3C, . . . 2CD3E, 2AB4C, . . . 2CD4E, 3AB2C, . . . 3CD2E, 3AB3C, . . . 3CD3E, 3AB4C, . . . 3CD4E, 4AB2C, . . . 4CD2E, 4AB3C, . . . 4CD3E, 4AB4C, . . . 4CD4E,

2A2B2C, . . . 2C2D2E, 2A3B2C, . . . 2C3D2E, 2A4B2C, . . . 2C4D2E, 3A2B2C, . . . 3C2D2E, 3A3B2C, . . . 3C3D2E, 3A4B2C, . . . 3C4D2E, 4A2B2C, . . . 4C2D2E, 4A3B2C, . . . 4C3D2E, 4A4B2C, . . . 4C4D2E,

2A2B3C, . . . 2C2D3E, 2A3B3C, . . . 2C3D3E, 2A4B3C, . . . 2C4D3E, 3A2B3C, . . . 3C2D3E, 3A3B3C, . . . 3C3D3E, 3A4B3C, . . . 3C4D3E, 4A2B3C, . . . 4C2D3E, 4A3B3C, . . . 4C3D3E, 4A4B3C, . . . 4C4D3E,

2A2B4C, . . . 2C2D4E, 2A3B4C, . . . 2C3D4E, 2A4B4C, . . . 2C4D4E, 3A2B4C, . . . 3C2D4E, 3A3B4C, . . . 3C3D4E, 3A4B4C, . . . 3C4D4E, 4A2B4C, . . . 4C2D4E, 4A3B4C, . . . 4C3D4E, 4A4B4C, 4A4B4D, 4A4B4E, 4B4C4D, 4B4C4E, 4C4D4E.

The number in front of a block refers to the relative amount of that block. Although this illustration uses whole number ratios of the amounts of the blocks, the relative amounts of blocks in or between block groups need not be in whole number increments, and need not even involve the same spacing between different amounts. Thus, a set of block groups could have blocks having relative amounts of, for example, 1, 1.25, 1.8, 2.4.

Note that all of the block groups are composed of exactly three different blocks, excluding combinations such as AAB, ADD, AAA. The excluded combinations have identify compositions of only one or two different blocks, which is outside the scope of this set of block groups. Note also that order does not matter. A block group having the identity composition of ABC is the same as a block group having the identity composition ACB. It should be understood that this illustration involves a set of block groups that includes all of the possible identity compositions of blocks and all possible amount compositions of blocks meeting the criteria of the block group set. Block group sets can also be composed of less than all of the possible identity compositions of blocks meeting the criteria of the block group set. A variable-amount block group set having less than all of the possible identity and/or amount compositions of blocks meeting the criteria of the block group set is still referred to as a variable-amount block group set.

C. Blocks

Blocks are molecules or moieties that can be associated with a carrier and which can be specifically detected. In particular, different blocks should be distinguishable upon detection. Blocks are generally composed of or comprise reporter signals. Reporter signals, which are described elsewhere herein, are molecules that can be preferentially fragmented, decomposed, reacted, derivatized, or otherwise modified or altered for detection. Blocks can be, for example, oligonucleotides, carbohydrates, synthetic polyamides, peptide nucleic acids, antibodies, ligands, proteins, peptides, haptens, zinc fingers, aptamers, mass labels, or reporter signals.

Blocks can be detected using any suitable detection technique. Many molecular detection techniques are known and can be used in the disclosed method. For example, blocks can be detected by nuclear magnetic resonance, electron paramagnetic resonance, surface enhanced raman scattering, surface plasmon resonance, fluorescence, phosphorescence, chemiluminescence, resonance raman, microwave, mass spectrometry, or any combination of these. Blocks can be separated and/or detected by, for example, mass spectrometry. Blocks can be distinguished temporally via different fluorescent, phosphorescent, or chemiluminescent emission lifetimes. The composition and characteristics of blocks should be matched with the chosen detection method.

Blocks can be isobaric blocks. Isobaric blocks have two key features. First, the isobaric blocks are used in sets where all the isobaric blocks in the set have similar properties (such as similar mass-to-charge ratios). The similar properties allow the isobaric blocks to be separated from other molecules lacking one or more of the properties. Second, all the isobaric blocks in a set can be fragmented, decomposed, reacted, derivatized, or otherwise modified to distinguish the different isobaric blocks in the set. The isobaric blocks can be, for example, fragmented to yield fragments of similar charge but different mass. Isobaric blocks are a form of reporter signal.

Blocks can be capable of being released by matrix-assisted laser desorption-ionization (MALDI) in order to be separated and identified (decoded) by time-of-flight (TOF) mass spectroscopy. For MALDI-TOF detection, the blocks can be peptide nucleic acids, where each block has a different mass to allow separation and separate detection in mass spectroscopy. For this purpose, it is useful to use combination of base composition and number of mass tags (e.g. the number of 8-amino-3,6-dioxaoctanoic monomers attached to the PNA (Griffin, T. J., W. Tang, and L. M. Smith, Genetic analysis by peptide nucleic acid affinity MALDI-TOF mass spectrometry. Nat Biotechnol, 1997. 15(12): p. 1368-72.)) to optimize the mass spectra for the set of blocks in a multiplex analysis.

Blocks can also be molecules capable of hybridizing specifically to a nucleic acid sequence. For this purpose peptide nucleic acid blocks can be used. Oligonucleotide or peptide nucleic acid blocks can have any arbitrary sequence. The only requirement is hybridization to nucleic acid sequences. The blocks can each be any length that supports specific and stable hybridization between the nucleic acid sequences and the blocks. For this purpose, a length of 10 to 35 nucleotides is preferred, with a length of 15 to 20 nucleotides long being most preferred.

1. Reporter Signals

Blocks are generally composed of or comprise reporter signals. Reporter signals are molecules that can be preferentially fragmented, decomposed, reacted, derivatized, or otherwise modified or altered for detection. Detection of the modified reporter signals can be accomplished with mass spectrometry. The disclosed reporter signals can be used in sets where members of a set have the same mass-to-charge ratio (m/z). This facilitates sensitive filtering or separation of reporter signals from other molecules based on mass-to-charge ratio. Reporter signals can have any structure that allows modification of the reporter signal and identification of the different modified reporter signals. Reporter signals can be composed such that at least one preferential bond rupture can be induced in the molecule. A set of reporter signals having nominally the same molecular mass and arbitrarily chosen internal fragmentation points may be constructed such that upon fragmentation each member of the set will yield unique correlated daughter fragments. For convenience, reporter signals that are fragmented, decomposed, reacted, derivatized, or otherwise modified for detection are referred to as fragmented reporter signals.

Useful reporter signals are made up of chains of subunits such as peptides, oligonucleotides, peptide nucleic acids, oligomers, carbohydrates, polymers, and other natural and synthetic polymers and any combination of these. Particularly useful chains are peptides, and are referred to herein as reporter signal peptides. Chains of subunits and subunits have a relationship similar to that of a polymers and mers. The mers are connected together to form a polymer. Likewise, subunits are connected together to form chains of subunits. Useful reporter signals are made up of chains of similar or related subunits. These are termed homochains or homopolymers. For example, nucleic acids are made up of phosphonucleosides and peptides are made up of amino acids.

Reporter signals can also be made up of heterochains or heteropolymers. A heterochain is a chain or a polymer where the subunits making up the chain are different types or the mers making up the polymer are different types. For example, a heterochain could be guanosine-alanine, which is made up of one nucleoside subunit and one amino acid subunit. It is understood that any combination of types of subunits can be used within the disclosed compositions, sets, and methods. Any molecule having the required properties can be used as a reporter signal. Useful reporter signals can be fragmented in tandem mass spectrometry.

Reporter signals can be used in sets where all the reporter signals in the set have similar physical properties. The similar (or common) properties allow the reporter signals to be distinguished and/or separated from other molecules lacking one or more of the properties. The reporter signals in a set can have, for example, the same mass-to-charge ratio (m/z). That is, the reporter signals in a set are isobaric. This allows the reporter signals (and/or the proteins to which they are attached) to be separated precisely from other molecules based on mass-to-charge ratio. The result of the filtering is a huge increase in the signal to noise ratio (S/N) for the system, allowing more sensitive and accurate detection. Such coordinated sets of reporter signals can be used within a set of block groups and/or within sets of detectors. In this regard, such sets of block groups (having blocks drawn from a set of reporter signals) can be used within sets of detectors.

Sets of reporter signals can have any number of reporter signals. For example, sets of reporter signals can have one, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, three hundred or more, four hundred or more, or five hundred or more different reporter signals. Although specific numbers of reporter signals and specific endpoints for ranges of the number of reporter signals are recited, each and every specific number of reporter signals and each and every specific endpoint of ranges of numbers of reporter signals are specifically contemplated, although not explicitly listed, and each and every specific number of reporter signals and each and every specific endpoint of ranges of numbers of reporter signals are hereby specifically described.

The sets of reporter signals can be made up of reporter signals that are made up of chains or polymers. The set of reporter signals can be homosets which means that the set is made up of one type of reporter signal or that the reporter signal is made up of homochains or homopolymers. The set of reporter signals can also be a heteroset which means that the set is made up of different reporter signals or of reporter signals that are made up of different types of chains or polymers. A special type of heteroset is one in which the set is made up of different homochains or homopolymers, for example one peptide chain and one nucleic acid chain. Another special type of heteroset is one where the chains themselves are heterochains or heteropolymers. Still another type of heteroset is one which is made up of both heterochains/heteropolymers and homochains/homopolymers.

A variety of different properties can be used as the common physical property used to separate reporter signals from other molecules lacking the common property. For example, other physical properties useful as common properties include mass, charge, isoelectric point, hydrophobicity, chromatography characteristics, and density. It is useful for the physical property shared by reporter signals in a set (and used to distinguish or separate the reporter signals from other molecules) to be an overall property of the reporter signal (for example, overall mass, overall charge, isoelectric point, overall hydrophobicity, etc.) rather than the mere presence of a feature or moiety (for example, an affinity tag, such as biotin). Such properties are referred to herein as “overall” properties (and thus, reporter signals in a set would be referred to as sharing a “common overall property”). It should be understood that reporter signals can have features and moieties, such as affinity tags, and that such features and moieties can contribute to the common overall property (by contributing mass, for example). However, such limited and isolated features and moieties would not serve as the sole basis of the common overall property.

A useful common overall property is the property of subunit isomers. This property occurs when a set of at least two reporter signals (which typically are made up of subunit chains which are in turn made up of subunits, for example, like the relationship between a polymer and the units that make up a polymer) is made up of subunit isomers, and the set could then be called subunit isomeric or isomeric for subunits. Subunits are discussed elsewhere herein, but reporter signals can be made up of any type of chain, such as peptides or nucleic acids or polymer (general) which are in turn made up of subunits for example amino acids and phosphonucleosides, and mers (general) respectively. Within each type of subunit there are typically multiple members that are all the same type of subunit, but differ. For example, within the subunit type “amino acids,” there are many members, for example, ala, tyr, and ser, or any other combination of amino acids.

When a set of reporter signals is subunit isomeric or is made up of subunit isomers this means that each individual of the set is a subunit isomer of every other individual subunit in the set. Isomer or isomeric means that the makeup of the subunits forming the subunit chain (that is, distribution or array) is the same but the overall connectivity of the subunits, forming the chain, is different. Thus, for example, a first reporter signal could be the chain, ala-ser-lys-gln, a second reporter signal could be the chain ala-lys-ser-gln, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, that is, each has one ala, one ser, one lys, and one gln, but each chain has a different connectivity. If, however, the set of reporter signals were made which contained the first, second, and third reporter signals the set would not be isomeric because the make up of each chain would not be the same because the first and second chains do not have a pro and the third chain does not have a gln.

Another illustration is the following: a first reporter signal could be the chain, ala-guanosine-lys-adenosine, a second reporter signal could be the chain ala-adenosine-lys-guanosine, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, that is, each has one ala, one guanosine, one lys, and one adenosine, but each chain has a different connectivity. If, however, the set of reporter signals were made which contained the first, second, and third reporter signals the set would not be isomeric because the makeup of each chain would not be the same because the first and second chains do not have a pro or a ser and the third chain does not have a guanosine or adenosine. This illustration shows that the sets can be made up of, or include, heterochains and still be considered subunit isomers.

It is useful if the common property of reporter signals is not an affinity tag. Nevertheless, even in such a case, reporter signals that otherwise have a common property may also include an affinity tag—and in fact may all share the same affinity tag—so long as another common property is present that can be (and, in some embodiments of the disclosed method, is) used to separate reporter signals sharing the common property from other molecules lacking the common property. With this in mind, it is useful that, if chromatography or other separation techniques are used to separate reporter signals based on the common property, the affinity be based on an overall physical property of the reporter signals and not on the presence of, for example, a feature or moiety such as an affinity tag. As used herein, a common property is a property shared by a set of components (such as reporter signals). That is, the components have the property “in common.” It should be understood that reporter signals in a set may have numerous properties in common. However, as used herein, the common properties of reporter signals referred to are only those used in the disclosed method to distinguish and/or separate the reporter signals sharing the common property from molecules that lack the common property.

Reporter signals in a set can be fragmented, decomposed, reacted, derivatized, or otherwise modified or altered to distinguish the different reporter signals in the set. The reporter signals can be fragmented to yield fragments of similar charge but different mass. The reporter signals can also be fragmented to yield fragments of different charge and mass. Such changes allow each reporter signal in a set to be distinguished by the different mass-to-charge ratios of the fragments of the reporter signals. This is possible since, although the unfragmented reporter signals in a set are isobaric, the fragments of the different reporter signals are not. Thus, a key feature of the disclosed reporter signals is that the reporter signals have a similarity of properties while the modified reporter signals are distinguishable.

Differential distribution of mass in the fragments of the reporter signals can be accomplished in a number of ways. For example, reporter signals of the same nominal structure (for example, peptides having the same amino acid sequence), can be made with different distributions of heavy isotopes, such as deuterium (²H), tritium (³H) ¹⁷O, ¹⁸O, ¹³C, or ¹⁴C; stable isotopes are preferred. All reporter signals in the set would have the same number of a given heavy isotope, but the distribution of these would differ for different reporter signals. An example of such a set of reporter signals is A*G*SLDPAGSLR, A*GSLDPAG*SLR, and AGSLDPA*G*SLR (SEQ ID NO:2), where the asterisk indicates at least one heavy isotope substituted amino acid. For a singly charged parent ion and, following fragmentation at the scissile DP bond, one predominantly charged daughter, there are three distinguishable primary daughter ions, PAGSLR⁺, PAG*SLR⁺, PA*G*SLR⁺ (amino acids 6-11 of SEQ ID NO:2).

Similarly, reporter signals of the same general structure (for example, peptides having the same amino acid sequence), can be made with different distributions of modifications or substituent groups, such as methylation, phosphorylation, sulphation, and use of seleno-methionine for methionine. All reporter signals in the set would have the same number of a given modification, but the distribution of these would differ for different reporter signals. An example of such a set of reporter signals is AGS*M*LDPAGSMLR, AGS*MLDPAGSM*LR, and AGS*MLDPAGS*M*LR (SEQ ID NO:3), where S* indicates phosphoserine rather than serine, and, M* indicates seleno-methionine rather than methionine. For a singly charged parent ion and, following fragmentation at the scissile DP bond, one predominantly charged daughter, there are three distinguishable primary daughter ions, PAGSMLR⁺, PAGSM*LR⁺, PAGS*M*LR⁺(amino acids 7-13 of SEQ ID NO:3).

Reporter signals of the same nominal composition (for example, made up of the same amino acids), can be made with different ordering of the subunits or components of the reporter signal. All reporter signals in the set would have the same number of subunits or components, but the distribution of these would be different for different reporter signals. An example of such a set of reporter signals is AGSLADPGSLR (SEQ ID NO:4), ALSLADPGSGR (SEQ ID NO:5), ALSLGDPASGR (SEQ ID NO:6). For a singly charged parent ion and, following fragmentation at the scissile DP bond, one predominantly charged daughter, there are three distinguishable primary daughter ions, PGSLR⁺ (amino acids 7-11 of SEQ ID NO:4), PGSGR⁺ (amino acids 7-11 of SEQ ID NO:5), PASGR⁺ (amino acids 7-11 of SEQ ID NO:6).

Reporter signals having the same nominal composition (for example, made up of the same amino acids), can be made with a labile or scissile bond at a different location in the reporter signal. All reporter signals in the set would have the same number and order of subunits or components. Where the labile or scissile bond is present between particular subunits or components, the order of subunits or components in the reporter signal can be the same except for the subunits or components creating the labile or scissile bond. Reporter signal peptides used in reporter signal fusions preferably use this form of differential mass distribution. An example of such a set of reporter signals is AGSLADPGSLR (SEQ ID NO:4), AGSDPLAGSLR (SEQ ID NO:7), ADPGSLAGSLR (SEQ ID NO:8). For a singly charged parent ion and, following fragmentation at the scissile DP bond, one predominantly charged daughter, there are three distinguishable primary daughter ions, PGSLR⁺ (amino acids 7-11 of SEQ ID NO:4), PLAGSLR⁺ (amino acids 5-11 of SEQ ID NO:7), PGSLAGSLR⁺ (amino acids 3-11 of SEQ ID NO:8).

Each of these modes can be combined with one or more of the other modes to produce differential distribution of mass in the fragments of the reporter signals. For example, different distributions of heavy isotopes can be used in reporter signals where a labile or scissile bond is placed in different locations. Different mass distribution can be accomplished in other ways. For example, reporter signals can have a variety of modifications introduced at different positions. Some examples of useful modifications include acetylation, methylation, phosphorylation, seleno-methionine rather than methionine, sulphation. Similar principles can be used to distribute charge differentially in reporter signals. Differential distribution of mass and charge can be used together in sets of reporter signals.

Reporter signals can also contain combinations of scissile bonds and labile bonds. This allows more combinations of distinguishable signals or to facilitate detection. For example, labile bonds may be used to release the isobaric fragments, and the scissile bonds used to decode the proteins.

Selenium substitution can be used to alter the mass of reporter signals. Selenium can substitute for sulfur in methionine, resulting in the modified amino acid selenomethionine. Selenium is approximately forty seven mass units larger than sulfur. Mass spectrometry may be used to identify peptides or proteins incorporating selenomethionine and methionine at a particular ratio. Small proteins and peptides with known selenium/sulfur ratio are preferably produced by chemical synthesis incorporating selenomethionine and methionine at the desired ratio. Larger proteins or peptides may be by produced from an E. coli expression system, or any other expression system that inserts selenomethionine and methionine at the desired ratio (Hendrickson et al., Selenomethionyl proteins procluded for analysis by multiwavelength anomalous diffraction (MAD): a vehicle for direct determination of three-dimensional structure. Embo J, 9(5):1665-72 (1990), Cowie and Cohen, Biosynthesis by Escherichia coli of active altered proteins containing selenium instead of sulfur. Biochimica et Biophysica Acta, 26:252 -261 (1957), and Oikawa et al., Metalloselenonein, the selenium analogue of metallothionein. synthesis and characterization of its complex with copper ions. Proc Natl Acad Sci USA, 88(8):3057-9 (1991).

Some forms of reporter signals can include one or more affinity tags. Such affinity tags can allow the detection, separation, sorting, or other manipulation of the labeled proteins, reporter signals, or reporter signal fragments based on the affinity tag. Such affinity tags are separate from and in addition to (not the basis of) the common properties of a set of reporter signals that allows separation of reporter signals from other molecules. Rather, such affinity tags serve the different purpose of allowing manipulation of a sample prior to or as a part of the disclosed method, not the means to separate reporter signals based on the common property. Reporter signals can have none, one, or more than one affinity tag. Where a reporter signal has multiple affinity tags, the tags on a given reporter signal can all be the same or can be a combination of different affinity tags. Affinity tags also can be used to distribute mass and/or charge differentially on reporter signals following the principles described above and elsewhere herein. Affinity tags can be used with reporter signals in a manner similar to the use of affinity labels as described in PCT Application WO 00/11208.

Peptide-DNA conjugates (Olejnik et al., Nucleic Acids Res., 27(23):4626-31 (1999)), synthesis of PNA-DNA constructs, and special nucleotides such as the photocleavable universal nucleotides of WO 00/04036 can be used as reporter signals in the disclosed method. Useful photocleavable linkages are also described by Marriott and Ottl, Synthesis and applications of heterobifunctional photocleavable cross-linking reagents, Methods Enzymol. 291:155-75 (1998).

Photocleavable bonds and linkages are useful in (and for use with) reporter signals because it allows precise and controlled fragmentation of the reporter signals (for subsequent detection) and precise and controlled release of reporter signals from detectors to which they are attached (and thus from analytes with which the detectors are associated). A variety of photocleavable bonds and linkages are known and can be adapted for use in and with reporter signals. Photocleavable amino acids are commercially available. For example, an Fmoc protected photocleavable slightly modified phenylalanine (Fmoc-D,L-β Phe(2-NO₂)) is available (Catalog Number 0011-F; Innovachem, Tucson, Ariz.). The introduction of the nitro group into the phenylalanine ring causes the amino acid to fragment under exposure to UV light (at a wavelength of approximately 350 nm). The nitrogen laser emits light at approximately 337 nm and can be used for fragmentation. The wavelength used will not cause significant damage to the rest of the peptide.

Fmoc synthesis is a common technique for peptide synthesis and Fmoc-derivative photocleavable amino acids can be incorporated into peptides using this technique. Although photocleavable amino acids are usable in and with any reporter signal, they are particularly useful in peptide reporter signals.

Use of photocleavable bonds and linkages in and with reporter signals can be illustrated with the following examples. Materials on a blank plastic substrate (for example, a Compact Disk (CD)) may be directly measured from that surface using a MALDI source ion trap. For example, a thin section of tissue sample, flash frozen, could be applied to the CD surface. A detector (for example, an antibody attached to a carrier with reporter signals attached via a photocleavable linkage) can be applied to the tissue surface. Recognition of specific components within the tissue allows for some of the detectors to associate (excess detectors are removed during subsequent wash steps). The reporter signal then can be released from the detector by applying a UV light and detected directly using the MALDI ion trap instrument. For example, a peptide of sequence CF*XXXXXDPXXXXXR (SEQ ID NO:1) (which contains a reporter signal) can be attached to the carrier in a detector using a disulfide bond linkage method. Exposure to the UV source of a MALDI laser will cleave the peptide at the modified phenylalanine, F*, releasing the XXXXXDPXXXXXR reporter signal (amino acids 3-15 of SEQ ID NO:1). The reporter signal subsequently can be fragmented at the DP bond and the charged fragment detected as described elsewhere herein.

A photocleavable linkage also can be incorporated into a reporter signal and used for fragmentation of the reporter signal in the disclosed methods. For example, a photocleavable amino acid (such as the photocleavable phenylalanine) can be incorporated at any desired position in a peptide reporter signal. A reporter signal such as XXXXXXF*XXXXXR containing photocleavable phenylalanine (F*) that is photocleavable. The reporter signal can then be fragmented using the appropriate wavelength of light and the charged fragment detected. When ionizing the reporter signal (from a surface, for example) for detection, a MALDI laser that does not cause significant photocleavage (for example, Er:YAG at 2.94 μm) can be used for ionization and a second laser (for example, Nitrogen at 337 nm) can be used to fragment the reporter signal. In this case XXXXXXFXXXXXR⁺ would be photocleaved to yield XXXXXR⁺. The second laser may intersect the reporter signal ion packet at any location. Modification to the vacuum system of a mass spectrometer for this purpose is straightforward.

Multiple photocleavable bonds and/or linkages can be used in or with the same reporter signals or detectors to achieve a variety of effects. For example, different photocleavable linkages that are cleaved by different wavelengths of light can be used in different parts of reporter signals or detectors to be cleaved at different stages of the method. Different fragmentation wavelengths allow sequential processing which enables, for example, the combinations of the release and fragmentation methods.

As an example, a peptide containing two photocleavable amino acids, Z (cleavage wavelength in the infrared) and F* (photocleavable phenylalanine, cleavage wavelength in UV) can be constructed of the form XZXXXXXXF*XXXXXXR where the amino terminus can be attached to a carrier or other molecule utilizing known chemistry. The reporter signal can be released from the detector by exposing the detector to an appropriate wavelength of light (infrared in this example), thus cleaving the bond at Z. Once the parent ion is selected and stored in the ion trap, the reporter signal can be fragmented by exposing it to an appropriate wavelength of light (UV in this example) to produce the daughter ion (XXXXXXR⁺) which can be detected and quantitated.

D. Specific Binding Molecules

A specific binding molecule is a molecule that interacts specifically with a particular molecule or moiety. The molecule or moiety that interacts specifically with a specific binding molecule is referred to herein as an analyte. Useful analytes are proteins and peptides. It is to be understood that the term analyte refers to both separate molecules and to portions of such molecules, such as an epitope of a protein, that interacts specifically with a specific binding molecule. Antibodies, either member of a receptor/ligand pair, synthetic polyamides (Dervan and Burli, Sequence-specific DNA recognition bypolyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are examples of specific binding molecules, useful as the affinity portion of a reporter binding molecule.

A specific binding molecule that interacts specifically with a particular analyte is said to be specific for that analyte. For example, where the specific binding molecule is an antibody that associates with a particular antigen, the specific binding molecule is said to be specific for that antigen. The antigen is the analyte. A detector containing the specific binding molecule can also be referred to as being specific for a particular analyte. Specific binding molecules can be antibodies, ligands, binding proteins, receptor proteins, haptens, aptamers, carbohydrates, synthetic polyamides, peptide nucleic acids, or oligonucleotides. Useful binding proteins are DNA binding proteins. Useful DNA binding proteins are zinc finger motifs, leucine zipper motifs, helix-turn-helix motifs. These motifs can be combined in the same specific binding molecule.

Antibodies useful as specific binding molecules, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

One form of specific binding molecule is an oligonucleotide or oligonucleotide derivative. Such specific binding molecules are designed for and used to detect specific nucleic acid sequences. Thus, the analyte for oligonucleotide specific binding molecules are nucleic acid sequences. The analyte can be a nucleotide sequence within a larger nucleic acid molecule. An oligonucleotide specific binding molecule can be any length that supports specific and stable hybridization between the reporter binding probe and the analyte. For this purpose, a length of 10 to 40 nucleotides is preferred, with an oligonucleotide specific binding molecule 16 to 25 nucleotides long being most preferred. It is useful for the oligonucleotide specific binding molecule to peptide nucleic acid. Peptide nucleic acid forms a stable hybrid with DNA. This allows a peptide nucleic acid specific binding molecule to remain firmly adhered to the target sequence during subsequent amplification and detection operations.

This useful effect can also be obtained with oligonucleotide specific binding molecules by making use of the triple helix chemical bonding technology described by Gasparro et al., Nucleic Acids Res., 22(14):2845-2852 (1994). Briefly, the oligonucleotide specific binding molecule is designed to form a triple helix when hybridized to a target sequence. This is accomplished generally as known, preferably by selecting either a primarily homopurine or primarily homopyrimidine target sequence. The matching oligonucleotide sequence which constitutes the specific binding molecule will be complementary to the selected target sequence and thus be primarily homopyrimidine or primarily homopurine, respectively. The specific binding molecule (corresponding to the triple helix probe described by Gasparro et al.) contains a chemically linked psoralen derivative. Upon hybridization of the specific binding molecule to a target sequence, a triple helix forms. By exposing the triple helix to low wavelength ultraviolet radiation, the psoralen derivative mediates cross-linking of the probe to the target sequence.

E. Analytes

The disclosed methods make use of analytes generally as objects of detection, measurement and/or analysis. Analytes can be any molecule or portion of a molecule that is to be detected, measured, or otherwise analyzed. An analyte need not be a physically separate molecule, but may be a part of a larger molecule. Analytes include biological molecules, organic molecules, chemicals, compositions, and any other molecule or structure to which the disclosed method can be adapted. It should be understood that different forms of the disclosed method are more suitable for some types of analytes than other forms of the method. Analytes are also referred to as target molecules.

Useful analytes are biological molecules. Biological molecules include but are not limited to proteins, peptides, enzymes, amino acid modifications, protein domains, protein motifs, nucleic acid molecules, nucleic acid sequences, DNA, RNA, mRNA, cDNA, metabolites, carbohydrates, and nucleic acid motifs. As used herein, “biological molecule” and “biomolecule” refer to any molecule or portion of a molecule or multi-molecular assembly or composition, that has a biological origin, is related to a molecule or portion of a molecule or multi-molecular assembly or composition that has a biological origin. Biomolecules can be completely artificial molecules that are related to molecules of biological origin.

Although reference is made above and elsewhere herein to detection of a “protein” or “proteins,” the disclosed method and compositions encompass proteins, peptides, and fragments of proteins or peptides. Thus, reference to a protein herein is intended to refer to proteins, peptides, and fragments of proteins or peptides unless the context clearly indicates otherwise.

F. Analyte Samples

Any sample from any source can be used with the disclosed method. In general, analyte samples should be samples that contain, or may contain, analytes. Examples of suitable analyte samples include cell samples, tissue samples, cell extracts, components or fractions purified from another sample, environmental samples, culture samples, tissue samples, bodily fluids, and biopsy samples. Numerous other sources of samples are known or can be developed and any can be used with the disclosed method. Useful analyte samples for use with the disclosed method are samples of cells and tissues. Analyte samples can be complex, simple, or anywhere in between. For example, an analyte sample may include a complex mixture of biological molecules (a tissue sample, for example), an analyte sample may be a highly purified protein preparation, or a single type of molecule.

G. Protein Samples

Any sample from any source can be used with the disclosed method. In general, protein samples should be samples that contain, or may contain, protein molecules. Examples of suitable protein samples include cell samples, tissue samples, cell extracts, components or fractions purified from another sample, environmental samples, biofilm samples, culture samples, tissue samples, bodily fluids, and biopsy samples. Numerous other sources of samples are known or can be developed and any can be used with the disclosed method. Useful protein samples for use with the disclosed method are samples of cells and tissues. Protein samples can be complex, simple, or anywhere in between. For example, a protein sample may include a complex mixture of proteins (a tissue sample, for example), a protein sample may be a highly purified protein preparation, or a single type of protein.

H. Capture Arrays

A capture array (also referred to herein as an array) includes a plurality of capture tags immobilized on a solid-state substrate, preferably at identified or predetermined locations on the solid-state substrate. In this context, plurality of capture tags refers to a multiple capture tags each having a different structure. Each predetermined location on the array (referred to herein as an array element) can have one type of capture tag (that is, all the capture tags at that location have the same structure). Each location will have multiple copies of the capture tag. The spatial separation of capture tags of different structure in the array allows separate detection and identification of analytes that become associated with the capture tags. If a block group is detected at a given location in a capture array, it indicates that the analyte corresponding to that array element was present in the target sample.

Solid-state substrates for use in capture arrays can include any solid material to which capture tags can be coupled, directly or indirectly. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, polyamino acids, controlled release polymers, gels, insoluble polymers, bioerodible polymers, resins, matrices, fibers, chromatography supports, hydrogels, polymers, plastics, glass, mica, gold, beads, microbeads, nanobeads, microspheres, nanospheres, particles, microparticles, nanoparticles, silicon, gallium arsenide, organic and inorganic metals, semiconductors, and insulators. Solid-state substrates can have any useful form including films or membranes, beads, bottles, dishes, disks, compact disks, fibers, optical fibers, woven fibers, polymers, shaped polymers, particles, probes, tips, bars, pegs, plugs, rods, sleeves, wires, filaments, tubes, ropes, tentacles, tethers, chains, capillaries, vessels, walls, edges, corners, seals, channels, lips, lattices, trellises, grids, arrays, knobs, steps, arms, teeth, cords, surfaces, layers, and thin films. A useful form for a solid-state substrate is a compact disk.

Although preferred, it is not required that a given capture array be a single unit or structure. The set of capture tags may be distributed over any number of solid supports. For example, at one extreme, each capture tag may be immobilized in a separate reaction tube or container. Arrays may be constructed upon non permeable or permeable supports of a wide variety of support compositions such as those described above. The array spot sizes and density of spot packing vary over a tremendous range depending upon the process(es) and material(s) used.

Methods for immobilizing antibodies and other proteins to substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A useful attachment agent is glutaraldehyde. These and other attachment agents, as well as methods for their use in attachment, are described in Protein immobilization: fundamentals and applications, Richard F. Taylor, ed. (M.

Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

Methods for immobilization of oligonucleotides to solid-state substrates are well established. Oligonucleotide capture tags can be coupled to substrates using established coupling methods. For example, suitable attachment methods are described by Pease et al., Proc. Natl. Acad. Sci. USA 91(11):5022-5026 (1994), Khrapko et al., Mol Biol (Mosk) (USSR) 25:718-730 (1991), U.S. Pat. No. 5,871,928 to Fodor et al., U.S. Pat. No. 5,654,413 to Brenner, U.S. Pat. No. 5,429,807, and U.S. Pat. No. 5,599,695 to Pease et al. A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Planar array technology has been utilized for many years (Shalon, D., S. J. Smith, and P. O. Brown, A DNA microarray system for analyzing complex DNA samples using two-color fluorescent probe hybridization. Genome Res, 1996. 6(7): p. 639-45, Singh-Gasson, S., et al., Maskless fabrication of light-directed oligonucleotide microarrays using a digital micromirror array. Nat Biotechnol, 1999. 17(10): p. 974-8, Southern, E. M., U. Maskos, and J. K. Elder, Analyzing and comparing nucleic acid sequences by hybridization to arrays of oligonucleotides: evaluation using experimental models. Genomics, 1992. 13(4): p. 1008-17, Nizetic, D., et al., Construction, arraying, and high-density screening of large insert libraries of human chromosomes X and 21: their potential use as reference libraries. Proc Natl Acad Sci USA, 1991. 88(8): p. 3233-7, Van Oss, C. J., R. J. Good, and M. K. Chaudhury, Mechanism of DNA (Southern) and protein (Western) blotting on cellulose nitrate and other membranes. J Chromatogr, 1987.391(1): p.53-65, Ramsay, G., DNA chips: state-of-the art. Nat Biotechnol, 1998. 16(1): p. 40-4, Schena, M., et al., Parallel human genome analysis: microarray-based expression monitoring of 1000 genes. Proc Natl Acad Sci USA, 1996. 93(20): p. 10614-9, Lipshutz, R. J., et al., High density synthetic oligonucleotide arrays. Nat Genet, 1999. 21(1 Suppl): p. 20-4, Pease, A. C., et al., Light-generated oligonucleotide arrays for rapid DNA sequence analysis. Proc Natl Acad Sci USA, 1994. 91(11): p. 5022-6, Maier, E., et al., Application of robotic technology to automated sequence fingerprint analysis by oligonucleotide hybridisation. J Biotechnol, 1994. 35(2-3): p. 191-203, Vasiliskov, A. V., et al., Fabrication of microarray of gel-immobilized compounds on a chip by copolymerization. Biotechniques, 1999. 27(3): p. 592-4, 596-8, 600 passim, and Yershov, G., et al., DNA analysis and diagnostics on oligonucleotide microchips. Proc Natl Acad Sci USA, 1996. 93(10): p. 4913-8).

Oligonucleotide capture tags in arrays can also be designed to have similar hybrid stability. This would make hybridization of fragments to such capture tags more efficient and reduce the incidence of mismatch hybridization. The hybrid stability of oligonucleotide capture tags can be calculated using known formulas and principles of thermodynamics (see, for example, Santa Lucia et al., Biochemistry 35:3555-3562 (1996); Freier et al., Proc. Natl Acad. Sci. USA 83:9373-9377 (1986); Breslauer et al., Proc. Natl. Acad. Sci. USA 83:3746-3750 (1986)). The hybrid stability of the oligonucleotide capture tags can be made more similar (a process that can be referred to as smoothing the hybrid stabilities) by, for example, chemically modifying the capture tags (Nguyen et al., Nucleic Acids Res. 25(1 5):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6):1492-1498 (1999); Wood et al., Proc. Natl. Acad. Sci. USA 82(6):1585-1588 (1985)).

Another means of smoothing hybrid stability of the oligonucleotide capture tags is to vary the length of the capture tags. This would allow adjustment of the hybrid stability of each capture tag so that all of the capture tags had similar hybrid stabilities (to the extent possible). Since the addition or deletion of a single nucleotide from a capture tag will change the hybrid stability of the capture tag by a fixed increment, it is understood that the hybrid stabilities of the capture tags in a capture array will not be equal. For this reason, similarity of hybrid stability as used herein refers to any increase in the similarity of the hybrid stabilities of the capture tags (or, put another way, any reduction in the differences in hybrid stabilities of the capture tags).

The efficiency of hybridization and ligation of oligonucleotide capture tags to sample fragments can also be improved by grouping capture tags of similar hybrid stability in sections or segments of a capture array that can be subjected to different hybridization conditions. In this way, the hybridization conditions can be optimized for particular classes of capture tags.

I. Capture Tags

A capture tag is any compound that can be used to capture or separate compounds or complexes having the capture tag. A capture tag can be a compound that interacts specifically with a particular molecule or moiety. The molecule or moiety that interacts specifically with a capture tag can be an analyte. It is to be understood that the term analyte refers to both separate molecules and to portions of such molecules, such as an epitope of a protein, that interacts specifically with a capture tag. Antibodies, either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are examples of capture tags.

A capture tag that interacts specifically with a particular analyte is said to be specific for that analyte. For example, where the capture tag is an antibody that associates with a particular antigen, the capture tag is said to be specific for that antigen. The antigen is the analyte. Capture tags can be antibodies, ligands, binding proteins, receptor proteins, haptens, aptamers, carbohydrates, synthetic polyamides, peptide nucleic acids, or oligonucleotides. Useful binding proteins are DNA binding proteins. Useful DNA binding proteins are zinc finger motifs, leucine zipper motifs, helix-turn-helix motifs. These motifs can be combined in the same capture tag.

Antibodies useful as the affinity portion of reporter binding agents, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).,

One form of capture tag is an oligonucleotide or oligonucleotide derivative. Such capture tags are designed for and used to detect specific nucleic acid sequences. Thus, the analyte for oligonucleotide capture tags are nucleic acid sequences. The analyte can be a nucleotide sequence within a larger nucleic acid molecule. An oligonucleotide capture tag can be any length that supports specific and stable hybridization between the capture tag and the analyte. For this purpose, a length of 10 to 40 nucleotides is preferred, with an oligonucleotide capture tag 16 to 25 nucleotides long being most preferred. It is useful for the oligonucleotide capture tag to be peptide nucleic acid. Peptide nucleic acid forms a stable hybrid with DNA. This allows a peptide nucleic acid capture tag to remain firmly adhered to the target sequence during subsequent amplification and detection operations.

This useful effect can also be obtained with oligonucleotide capture tags by making use of the triple helix chemical bonding technology described by Gasparro et al., Nucleic Acids Res., 22(14):2845-2852 (1994). Briefly, the oligonucleotide capture tag is designed to form a triple helix when hybridized to a target sequence. This is accomplished generally as known, preferably by selecting either a primarily homopurine or primarily homopyrimidine target sequence. The matching oligonucleotide sequence which constitutes the capture tag will be complementary to the selected target sequence and thus be primarily homopyrimidine or primarily homopurine, respectively. The capture tag (corresponding to the triple helix probe described by Gasparro et al.) contains a chemically linked psoralen derivative. Upon hybridization of the capture tag to a target sequence, a triple helix forms. By exposing the triple helix to low wavelength ultraviolet radiation, the psoralen derivative mediates cross-linking of the probe to the target sequence.

J. Sample Arrays

A sample array includes a plurality of samples (for example, expression samples, tissue samples, protein samples) immobilized on a solid-state substrate, preferably at identified or predetermined locations on the solid-state substrate. Each predetermined location on the sample array (referred to herein as an sample array element) can have one type of sample. The spatial separation of different samples in the sample array allows separate detection and identification of detectors (or block groups or blocks) that become associated with the samples. If a detector is detected at a given location in a sample array, it indicates that the analyte corresponding to that detector was present in the sample corresponding to that sample array element.

Solid-state substrates for use in sample arrays can include any solid material to which samples can be adhered, directly or indirectly. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, polyamino acids, controlled release polymers, gels, insoluble polymers, bioerodible polymers, resins, matrices, fibers, chromatography supports, hydrogels, polymers, plastics, glass, mica, gold, beads, microbeads, nanobeads, microspheres, nanospheres, particles, microparticles, nanoparticles, silicon, gallium arsenide, organic and inorganic metals, semiconductors, and insulators. Solid-state substrates can have any useful form including films or membranes, beads, bottles, dishes, disks, compact disks, fibers, optical fibers, woven fibers, polymers, shaped polymers, particles, probes, tips, bars, pegs, plugs, rods, sleeves, wires, filaments, tubes, ropes, tentacles, tethers, chains, capillaries, vessels, walls, edges, corners, seals, channels, lips, lattices, trellises, grids, arrays, knobs, steps, arms, teeth, cords, surfaces, layers, and thin films. A useful form for a solid-state substrate is a compact disk.

Although preferred, it is not required that a given sample array be a single unit or structure. The set of samples may be distributed over any number of solid supports. For example, at one extreme, each sample may be immobilized in a separate reaction tube or container. Sample arrays may be constructed upon non permeable or permeable supports of a wide variety of support compositions such as those described above. The array spot sizes and density of spot packing vary over a tremendous range depending upon the process(es) and material(s) used. Methods for adhering or immobilizing samples and sample components to substrates are well established.

A useful form of sample array is a tissue arrays, where there are small tissue samples on a substrate. Such tissue microarrays exist, and are used, for example, in a cohort to study breast cancer. The disclosed method can be used, for example, to probe multiple analytes in multiple samples. Sample arrays can be, for example, labeled with different reporter signals, the whole support then introduced into source region of a mass spec, and sampled by MALDI.

K. Decoding Tags

Decoding tags are any molecule or moiety that can be associated with coding tags or reporter molecules, directly or indirectly. Decoding tags are associated with blocks to allow indirect association of the blocks with a detector. Decoding tags can be oligonucleotides, carbohydrates, synthetic polyamides, peptide nucleic acids, antibodies, ligands, proteins, haptens, zinc fingers, aptamers, or mass labels.

Useful decoding tags are molecules capable of hybridizing specifically to an oligonucleotide coding tag. Most useful are peptide nucleic acid decoding tags. Oligonucleotide or peptide nucleic acid decoding tags can have any arbitrary sequence. The only requirement is hybridization to coding tags. The decoding tags can each be any length that supports specific and stable hybridization between the coding tags and the decoding tags. For this purpose, a length of 10 to 35 nucleotides is preferred, with a decoding tag 15 to 20 nucleotides long being most preferred.

Blocks containing decoding tags can be capable of being released by matrix-assisted laser desorption-ionization (MALDI) in order to be separated and identified by time-of-flight (TOF) mass spectroscopy, or by another detection technique. A decoding tag may be any oligomeric molecule that can hybridize to a coding tag. For example, a decoding tag can be a DNA oligonucleotide, an RNA oligonucleotide, or a peptide nucleic acid (PNA) molecule. Useful decoding tags are PNA molecules.

L. Coding Tags

Coding tags are molecules or moieties with which decoding tags can associate. Coding tags can be any type of molecule or moiety that can serve as a target for decoding tag association. Useful coding tags are oligomers, oligonucleotides, or nucleic acid sequences. Coding tags can also be a member of a binding pair, such as streptavidin or biotin, where its cognate decoding tag is the other member of the binding pair. Coding tags can also be designed to associate directly with some types of blocks. For example, oligonucleotide coding tags can be designed to interact directly with peptide nucleic acid blocks (which are blocks composed of peptide nucleic acid), such as peptide nucleic acid reporter signals.

The oligomeric base sequences of oligomeric coding tags can include RNA, DNA, modified RNA or DNA, modified backbone nucleotide-like oligomers such as peptide nucleic acid, methylphosphonate DNA, and 2′-O-methyl RNA or DNA. Oligomeric or oligonucleotide coding tags can have any arbitrary sequence. The only requirement is association with decoding tags (preferably by hybridization). In the disclosed method, multiple coding tags can become associated with a single carrier or analyte.

Oligonucleotide coding tags can each be any length that supports specific and stable hybridization between the coding tags and the decoding tags. For this purpose, a length of 10 to 35 nucleotides is preferred, with a coding tag 15 to 20 nucleotides long being most preferred.

The branched DNA for use as a carrier is generally known (Urdea, Biotechnology 12:926-928 (1994), and Horn et al., Nucleic Acids Res 23:4835-4841 (1997)). As used herein, the tail of a branched DNA molecule refers to the portion of a branched DNA molecule that is designed to interact with the analyte. The tail is a specific binding molecule. In general, each branched DNA molecule should have only one tail. The branches of the branched DNA (also referred to herein as the arms of the branched DNA) can contain coding tag sequences. Oligonucleotide dendrimers (or dendrimeric DNA) are also generally known (Shchepinov et al., Nucleic Acids Res. 25:4447-4454 (1997), and Orentas et al., J. Virol. Methods 77:153-163 (1999)). As used herein, the tail of an oligonucleotide dendrimer refers to the portion of a dendrimer that is designed to interact with the analyte. In general, each dendrimer should have only one tail. The dendrimeric strands of the dendrimer are referred to herein as the arms of the oligonucleotide dendrimer and can contain coding tag sequences.

M. Reporter Molecules

Reporter molecules are molecules that combine a specific binding molecule with a coding tag. The specific binding molecule and coding tag can be covalent coupled or tethered to each other. As used herein, molecules are coupled when they are covalent joined, directly or indirectly. One form of indirect coupling is via a linker molecule. The coding tag can be coupled to the specific binding molecule by any of several established coupling reactions. For example, Hendrickson et al., Nucleic Acids Res., 23(3):522-529 (1995) describes a suitable method for coupling oligonucleotides to antibodies. These reporter molecules are the functional equivalents of the reporter molecules described in PCT Application WO 00/68434 and can be used as described therein in combination with the compositions and methods described herein.

As used herein, a molecule is said to be tethered to another molecule when a loop of (or from) one of the molecules passes through a loop of (or from) the other molecule. The two molecules are not covalently coupled when they are tethered. Tethering can be visualized by the analogy of a closed loop of string passing through the hole in the handle of a mug. In general, tethering is designed to allow one or both of the molecules to rotate freely around the loop.

N. Affinity Tags

An affinity tag is any compound that can be used to separate compounds or complexes having the affinity tag from those that do not. An affinity tag can be a compound, such as a ligand or hapten, that associates or interacts with another compound, such as ligand-binding molecule or an antibody. It is also useful for such interaction between the affinity tag and the capturing component to be a specific interaction, such as between a hapten and an antibody or a ligand and a ligand-binding molecule. Affinity tags can be antibodies, ligands, binding proteins, receptor proteins, haptens, aptamers, carbohydrates, synthetic polyamides, or oligonucleotides. Preferred binding proteins are DNA binding proteins. Useful DNA binding proteins are zinc finger motifs, leucine zipper motifs, helix-turn-helix motifs. These motifs can be combined in the same specific binding molecule.

Affinity tags, described in the context of nucleic acid probes, are described by Syvnen et al., Nucleic Acids Res., 14:5037 (1986). Useful affinity tags include biotin, which can be incorporated into nucleic acids. In the disclosed method, affinity tags incorporated into reporter signals can allow the reporter signals to be captured by, adhered to, or coupled to a substrate. Such capture allows separation of reporter signals from other molecules, simplified washing and handling of reporter signals, and allows automation of all or part of the method.

Zinc fingers can also be used as affinity tags. Properties of zinc fingers, zinc finger motifs, and their interactions, are described by Nardelli et al., Zinc finger—DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran, S. and J. Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

Capturing detectors or blocks on a substrate, if desired, may be accomplished in several ways. In one embodiment, affinity docks are adhered or coupled to the substrate. Affinity docks are compounds or moieties that mediate adherence of a detector or block by associating or interacting with an affinity tag on the detector or block. Affinity docks immobilized on a substrate allow capture of the detectors or blocks on the substrate. Such capture provides a convenient means of washing away molecules that might interfere with subsequent steps. Captured detectors or blocks can also be released from the substrate. This can be accomplished by dissociating the affinity tag or by breaking a photocleavable linkage between, for example, the detector or block and the substrate, or between the block and the carrier.

Substrates for use in the disclosed method can include any solid material to which the disclosed components can be adhered or coupled. Examples of substrates include, but are not limited to, materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, Teflon, fluorocarbons, nylon, silicon rubber, polyanhydrides, polyglycolic acid, polylactic acid, polyorthoesters, polypropylfumerate, collagen, glycosaminoglycans, polyamino acids, controlled release polymers, gels, insoluble polymers, bioerodible polymers, resins, matrices, fibers, chromatography supports, hydrogels, polymers, plastics, glass, mica, gold, beads, microbeads, nanobeads, microspheres, nanospheres, particles, microparticles, nanoparticles, silicon, gallium arsenide, organic and inorganic metals, semiconductors, and insulators. Solid-state substrates can have any useful form including films or membranes, beads, bottles, dishes, disks, compact disks, fibers, optical fibers, woven fibers, polymers, shaped polymers, particles, probes, tips, bars, pegs, plugs, rods, sleeves, wires, filaments, tubes, ropes, tentacles, tethers, chains, capillaries, vessels, walls, edges, corners, seals, channels, lips, lattices, trellises, grids, arrays, knobs, steps, arms, teeth, cords, surfaces, layers, and thin films.

O. Mass Spectrometers

The disclosed methods can make use of mass spectrometers for analysis of blocks such as reporter signals, and altered forms of blocks or reporters signals. Mass spectrometers are generally available and such instruments and their operations are known to those of skill in the art. Fractionation systems integrated with mass spectrometers are commercially available, exemplary systems include liquid chromatography (LC) and capillary electrophoresis (CE).

The principle components of a mass spectrometer include: (a) one or more sources, (b) one or more analyzers and/or cells, and (c) one or more detectors. Types of sources include Electrospray Ionization (ESI) and Matrix Assisted Laser Desorption Ionization (MALDI). Types of analyzers and cells include quadrupole mass filter, hexapole collision cell, ion cyclotron trap, and Time-of-Flight (TOF). Types of detectors include Multichannel Plates (MCP) and ion multipliers. A useful mass spectrometer for use with the disclosed method is described by Krutchinsky et al., Rapid Automatic Identification of Proteins Utilizing a Novel MALDI-Ion Trap Mass Spectrometer, Abstract of the 49^{th} ASMS Conference on Mass Spectrometry and Allied Topics (May 27-31, 2001), The Rockefeller University, New York, N.Y.

Mass spectrometers with more than one analyzer/cell are known as tandem mass spectrometers. There are two types of tandem mass spectrometers, as well as hybrids and combinations of these types: “tandem in space” spectrometers and “tandem in time” spectrometers. Tandem mass spectrometers where the ions traverse more than one analyzer/cell are known as tandem in space mass spectrometers. Tandem in space spectrometers utilize spatially ordered elements and act upon the ions in turn as the ions pass through each element. Tandem mass spectrometers where the ions remain primarily in one analyzer/cell are known as tandem in time mass spectrometers. Tandem in time spectrometers utilize temporally ordered manipulations on the ions as the ions are contained in a space. Hybrid systems and combinations of these types are known. The ability to select a particular mass-to-charge ratio of interest in a mass analyzer is typically characterized by the resolution (reported as the centroid mass-to-charge divided by the full width at half maximum of the selected ions of interest). Thus resolution is an indicator of the narrowness of the ion mass-to-charge distribution passed through the analyzer to the detector. Reference to such resolution is generally noted herein by referring to the ability of a mass spectrometer to pass only a narrow range of mass-to-charge ratios.

A useful form of mass spectrometer for use in the disclosed methods is a tandem mass spectrometer, such as a tandem in space tandem mass spectrometer. As an example of the use of a tandem in space class of instrument, isobaric reporter signals can be first passed through a filtering quadrupole, the reporter signals are fragmented (preferably in a collision cell), and the fragments are distinguished and detected in a time-of-flight (TOF) stage. In such an instrument the sample is ionized in the source (for example, in a MALDI ion source) to produce charged ions. It is useful for the ionization conditions to be such that primarily a singly charged parent ion is produced. A first quadrupole, Q0, is operated in radio frequency (RF) mode only and acts as an ion guide for all charged particles. The second quadrupole, Q1, is operated in RF+DC mode to pass only a narrow range of mass-to-charge ratios (that includes the mass-to-charge ratio of the reporter signals). This quadrupole selects the mass-to-charge ratio of interest. Quadrupole Q2, surrounded by a collision cell, is operated in RF only mode and acts as ion guide. The collision cell surrounding Q2 can be filled to appropriate pressure with a gas to fracture the input ions by collisionally induced dissociation when fragmentation of the reporter signals is desired. The collision gas can be chemically inert, but reactive gases can also be used. Useful molecular systems utilize reporter signals that contain scissile bonds, labile bonds, or combinations, such that these bonds will be preferentially fractured in the Q2 collision cell.

Tandem instruments capable of MS^{N} can be used with the disclosed method. As an example consider; a method where one selects a set of molecules using a first stage filter (MS), photocleaves these molecules to yield a set of reporter signals, selects these reporter signals using a second stage (MS/MS), alters these reporter signals by collisional fragmentation, detects by time of flight (MS3). Many other combinations are possible and the disclosed method can be adapted for use with such systems. For example, extension to more stages, or analysis of reporter signal fragments is within the skill of those in the art.

### Methods

The disclosed detectors can be used in a method of detecting multiple analytes in a sample in a single assay. The method is based on encoding target molecules with signals followed by decoding of the encoded signal. This encoding/decoding uncouples the detection of a target molecule from the chemical and physical properties of the target molecule. In basic form, the disclosed method involves association of one or more detectors with one or more target samples—where the detector comprises a specific binding molecule, a carrier, and a block group composed of blocks—and detection of the block groups via detection of the blocks. The detectors associate with target molecules in the target sample(s) via the specific binding molecule. Generally, the detectors correspond to one or more target molecules, and the block groups correspond to one or more detectors. Thus, detection of particular block groups indicates the presence of the corresponding detectors. In turn, the presence of particular detectors indicates the presence of the corresponding target molecules.

This indirect detection uncouples the detection of target molecules from the chemical and physical properties of the target molecules by interposing block groups that essentially can have any arbitrary chemical and physical properties. In particular, block groups (and the blocks of which they are composed) can have specific properties useful for detection, and block groups and blocks within an assay can have highly ordered or structured relationships with each other. It is the (freely chosen) properties of the block groups and blocks, rather than the (take them as they are) properties of the target molecules that matters at the point of detection.

Useful blocks are isobaric blocks and reporter signals (which can also be isobaric). Isobaric blocks have two key features. First, the isobaric blocks are used in sets where all the isobaric blocks in the set have similar properties (such as similar mass-to-charge ratios). The similar properties allow the isobaric blocks to be separated from other molecules lacking one or more of the properties. Second, all the isobaric blocks in a set can be fragmented, decomposed, reacted, derivatized, or otherwise modified to distinguish the different isobaric blocks in the set. The isobaric blocks can be fragmented to yield fragments of similar charge but different mass.

The disclosed compositions and methods can be usefully combined with the system of multiple tag analysis described in PCT Application WO 00/68434. In basic terms, multiple tag analysis involves association of one or more reporter molecules with one or more target samples, association of one or more decoding tags with the reporter molecules, and detection of the decoding tags. The reporter molecules associate with target molecules in the target sample(s). Reporter molecules are composed of a specific binding molecule (for specific interaction with target molecules) and a reporter tag (for specific interaction with decoding tags). Generally, the reporter molecules correspond to one or more target molecules, and the decoding tags correspond to one or more reporter molecules. Thus, detection of particular decoding tags indicates the presence of the corresponding reporter molecules. In turn, the presence of particular reporter molecules indicates the presence of the corresponding target molecules. Multiple tag analysis is fully described in PCT Application WO 00/68434.

Following association of detectors with analytes, the disclosed methods can involve two basic steps. A filtering, selection, or separation step to separate blocks that are reporter signals from other molecules that may be present, and a detection step that distinguishes different reporter signals. The reporter signals can be distinguished and/or separated from other molecules based on some common property shared by the reporter signals but not present in most (or, preferably, all) other molecules present. The separated reporter signals are then treated and/or detected such that the different reporter signals are distinguishable. Useful forms of the disclosed method involve association of reporter signals with analytes of interest. Detection of the reporter signals results in detection of analytes with which the corresponding detectors are associated. Thus, the disclosed method is a general technique for labeling and detection of analytes.

A useful form of the disclosed method involves filtering of blocks that are isobaric reporter signals from other molecules based on mass-to-charge ratio, fragmentation of the reporter signals to produce fragments having different masses, and detection of the different fragments based on their mass-to-charge ratios. The method is best carried out using a tandem mass spectrometer. There are two types of tandem mass spectrometers, as well as hybrids and combinations of these types: “tandem in space” spectrometers and “tandem in time” spectrometers. Tandem in space spectrometers utilize spatially ordered elements and act upon the ions in turn as the ions pass through each element. Tandem in time spectrometers utilize temporally ordered manipulations on the ions as the ions are contained in a space. In a tandem in space class of instrument, the isobaric reporter signals are first passed through a filtering quadrupole, the reporter signals are fragmented (preferably in a collision cell), and the fragments are distinguished and detected in a time-of-flight (TOF) stage. In such an instrument the sample is ionized in the source (for example, in a MALDI) to produce charged ions. It is useful for the ionization conditions to be such that primarily a singly charged parent ion is produced. A first quadrupole, Q0, is operated in radio frequency (RF) mode only and acts as an ion guide for all charged particles. The second quadrupole, Q1, is operated in RF+DC mode to pass only a narrow range of mass-to-charge ratios (that includes the mass-to-charge ratio of the reporter signals). This quadrupole selects the mass-to-charge ratio of interest. Quadrupole Q2, surrounded by a collision cell, is operated in RF only mode and acts as ion guide. The collision cell surrounding Q2 can be filled to appropriate pressure with a gas to fracture the input ions by collisionally induced dissociation. The collision gas can be chemically inert, but reactive gases can also be used. Useful molecular systems utilize reporter signals that contain scissile bonds, labile bonds, or combinations, such that these bonds will be preferentially fractured in the Q2 collision cell.

The disclosed method is particularly well suited to the use of a MALDI-QqTOF mass spectrometer. The method enables highly multiplexed analyte detection, and very high sensitivity. Useful tandem mass spectrometers are described by Loboda et al., Design and Performance of a MALDI-QqTOF Mass Spectrometer, in 47th ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000). In such an instrument the sample is ionized in the source (MALDI, for example) to produce charged ions; it is useful for the ionization conditions to be such that primarily a singly charged parent ion is produced. First and third quadrupoles, Q0 and Q2, will be operated in RF only mode and will act as ion guides for all charged particles, second quadrupole Q1 will be operated in RF+DC mode to pass only a particular mass-to-charge (or, in practice, a narrow mass-to-charge range). This quadrupole selects the mass-to-charge ratio, (m/z), of interest. The collision cell surrounding Q2 can be filled to appropriate pressure with a gas to fracture the input ions by collisionally induced dissociation (normally the collision gas is chemically inert, but reactive gases are contemplated). Useful molecular systems utilize reporter signals that contain scissile bonds, labile bonds, or combinations, and these bonds will be preferentially fractured in the Q2 collision cell.

A MALDI source is useful for the disclosed method because it facilitates the multiplexed analysis of samples from heterogeneous environments such as arrays, beads, microfabricated devices, tissue samples, and the like. An example of such an instrument is described by Qin et al., A practical ion trap mass spectrometerfor the analysis of peptides by matrix-assisted laser desorption/ionization., Anal. Chem., 68:1784 -1791 (1996). For homogeneous assays electrospray ionization (ESI) sources will work very well. Electrospray ionization source instruments interfaced to LC systems are commercially available (for example, QSTAR from PE-SCIEX, Q-TOF from Micromass). It is of note that the ESI sources are operated such that they tend to produce multiply charged ions, doubly charged ions would be most common for ions in the disclosed method. Such doubly charged ions are well known in the art and present no limitation to the disclosed method. TOF analyzers and quadrupole analyzers are preferred detectors over sector analyzers. Tandem in time ion trap systems such as Fourier Transform Ion Cyclotron Resonance (FT-ICR) mass spectrometers also may be used with the disclosed method.

A number of elements contribute to the sensitivity of the disclosed method. The filter quadrupole, Q1, selects a narrow mass-to-charge ratio and discriminates against other mass-to-charge ions, significantly decreasing background from non germane ions. For example, for a sample containing a distribution of mass-to-charges of width 3000 Da, a mass-to-charge transmission window of 2 Da applied to this distribution can improve the signal to noise by at least a factor of 3000/2=1500. Once the parent ion is selected by quadrupole Q1, fragmentation of the parent ion, preferably into a single charged daughter ion, has the advantage over systems which fragment the parent into a number of daughter ions. For example, a parent fragmented into 20 daughter ions will yield signals that are on average 1/20^{th} the intensity of the parent ions. For a parent to single daughter system there will not be this signal dilution.

This preferred system for use with the disclosed method has a high duty cycle, and as such good statistics can be collected quickly. For the case where a single set of isobaric parents is used, the multiplexed detection is accomplished without having to scan the filter quadrupole (although such a scan is useful for single pass analysis of a complex protein sample with multiple labeled proteins). Electrospray sources can operate continuously, MALDI sources can operate at several kHz, quadrupoles operate continuously, and time of flight analyzers can capture the entire mass-to-charge region of interest at several kHz repetition rate. Thus, the overall system can acquire thousands of measurements per second. For throughput advantage in a multiplexed assay the time of flight analyzer has an advantage over a quadruple analyzer for the final stage because the time of flight analyzer detects all fragment ions in the same acquisition rather than requiring scanning (or stepping) over the ions with a quadrupole analyzer.

Instrumental improvements including addition of laser ports along the flight path to allow intersection of the proteins with additional laser(s) open additional fragmentation avenues through photochemical and photophysical processes (for example, selective bond cleavage, selective ionization). Use of lasers to fragment the proteins after the filter stage will enable the use of the very high throughput TOF-TOF instruments (50 kHz to 100 kHz systems).

The disclosed method is compatible with techniques involving cleavage, treatment, or fragmentation of a bulk sample in order to simplify the sample prior to introduction into the first stage of a multistage detection system. The disclosed method is also compatible with any desired sample, including raw extracts and fractionated samples.

In one form of the disclosed method, detectors (and thus, reporter signals that are the blocks making up the block groups on the detectors) are associated with analytes to be detected and/or quantitated. The specific binding molecule in the detector interacts with the analyte thus associating the detector (and the reporter signals) with the analyte. The disclosed method increases the sensitivity and accuracy of detection of an analyte of interest. Useful forms of the disclosed method make use of multistage detection systems to increase the resolution of the detection of molecules having very similar properties. The method involves at least two stages. The first stage is filtration or selection that allows passage or selection of reporter signals (that is, a subset of the molecules present), based upon intrinsic properties of the reporter signals, and discrimination against all other molecules. The subsequent stage(s) further separate(s) and/or detect(s) the reporter signals which were filtered in the first stage. A key facet of this method is that a multiplexed set of reporter signals will be selected by the filter and subsequently cleaved, decomposed, reacted, or otherwise modified to realize the identities and/or quantities of the reporter signals in further stages. There is a correspondence between the specific binding molecule and the detected daughter fragment.

Forms and Embodiments of the Disclosed Material and Methods

The disclosed compositions and methods can be further described and understood by the following descriptions of embodiments.

Disclosed is a method of detecting analytes, the method comprising associating one or more detectors with one or more target samples, wherein the detectors each comprise a specific binding molecule, a carrier, and a block group, wherein the block group comprises blocks, wherein the blocks comprise reporter signals, and detecting the block group. The reporter signals can have a common property, wherein the common property can allow the reporter signals to be distinguished or separated from molecules lacking the common property, wherein the reporter signals can be altered, wherein the altered forms of each reporter signal can be distinguished from every other altered form of reporter signal. The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. Alteration of the reporter signals also can alter their charge.

The common property can be mass-to-charge ratio, wherein the reporter signals can be altered by altering their charge, wherein the altered forms of the labeled proteins can be distinguished via differences in the mass-to-charge ratio of the altered forms of reporter signals. The block group can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The block group can comprise ten or more different reporter signals.

The reporter signals can be peptides, oligonucleotides, carbohydrates, polymers, oligopeptides, or peptide nucleic acids. The reporter signals can be associated with, or coupled to, specific binding molecules, wherein each reporter signal can be associated with, or coupled to, a different specific binding molecule. The reporter signals can be associated with, or coupled to, decoding tags, wherein each reporter signal can be associated with, or coupled to, a different decoding tag. The reporter signals can comprise peptides, wherein the peptides can have the same mass-to-charge ratio. The peptides can have the same amino acid composition. The peptides can have the same amino acid sequence. Each peptide can contain a different distribution of heavy isotopes. Each reporter signal peptide can contain a different distribution of substituent groups. Each peptide can have a different amino acid sequence. Each peptide can have a labile or scissile bond in a different location.

The reporter signals can be coupled to the proteins or peptides. The common property can allow the labeled proteins to be distinguished or separated from molecules lacking the common property. The common property need not be an affinity tag. One or more affinity tags can be associated with the reporter signals.

The blocks can have the same amount composition. The blocks need not all have the same amount composition. A plurality of detectors can be associated with the one or more target samples, wherein the block group of each detector can have a different composition of blocks. Each block group can have the same number of blocks. The block groups need not all have the same number of blocks. Each block group can have a different identity composition of blocks. The blocks can have the same amount composition. The blocks need not all have the same amount composition. Block groups that have the same identity composition of blocks can have different amount compositions of blocks.

The blocks can be peptide nucleic acids. The blocks can be capable of hybridizing specifically to a nucleic acid sequence. The length of the nucleic acid sequence can be between 10 and 35 nucleotides long. The length of the nucleic acid sequence can be between 15 and 20 nucleotides long. The blocks can be capable of being detected by a method selected from the group consisting of nuclear magnetic resonance, electron paramagnetic resonance, surface enhanced raman scattering, surface plasmon resonance, fluorescence, phosphorescence, chemiluminescence, resonance ram an, microwave, mass spectrometry, mass spectrometry electrophoresis chromatography, and any combination of these.

The blocks can be capable of being detected through MALDI-TOF spectroscopy. The blocks can be isobaric blocks. A plurality of detectors can be associated with one or more target samples, wherein the blocks of each detector can be different. All of the blocks of all of the detectors can have the same mass-to-charge ratio. The blocks can be altered by altering their mass, charge, or both, wherein the altered forms of the blocks can be distinguished via differences in the mass-to-charge ratio of the altered forms of the blocks.

The carrier can be selected from the group consisting of beads, liposomes, microparticles, nanoparticles, and branched polymer structures. The carrier can be a bead. The carrier can be a liposome or microbead. The liposomes can be unilamellar vesicles. The vesicles can have an average diameter of 150 to 300 nanometers. The liposome can have an internal diameter of 200 nanometers. The carrier can be a dendrimer. The dendrimer can be contacting a macromolecule selected from the group consisting of DNA, RNA, and PNA. The macromolecule can be an oligonucleotide between 20 and 300 nucleotides in length.

The specific binding molecule can be selected from the group consisting of antibodies, ligands, binding proteins, receptor proteins, haptens, aptamers, carbohydrates, synthetic polyamides, and oligonucleotides. The specific binding molecule can be a binding protein. The binding protein can be a DNA binding protein. The DNA binding protein can contain a motif selected from the group consisting of a zinc finger motif, leucine zipper motif, and helix-turn-helix motif.

The specific binding molecule can be an oligonucleotide. The oligonucleotide can be between 10 and 40 nucleotides in length. The oligonucleotide can be between 16 and 25 nucleotides in length. The oligonucleotide can be a peptide nucleic acid. The oligonucleotide can form a triple helix with the target sequence. The oligonucleotide can comprise a psoralen derivative capable of covalently attaching the oligonucleotide to the target sequence.

The specific binding molecule can be an antibody. The antibody can bind a protein. The blocks can be oligonucleotides, carbohydrates, synthetic polyamides, peptide nucleic acids, antibodies, ligands, proteins, haptens, zinc fingers, aptamers, mass labels, or any combination of these. The specific binding molecule and the carrier can be covalently linked. The carrier and the blocks can be covalently linked. The specific binding molecule and the carrier can be covalently linked. The specific binding molecule can comprise a first oligonucleotide and the carrier can comprise a second oligonucleotide which can hybridize to the first oligonucleotide. The first oligonucleotide can be conjugated to an antibody which binds a protein.

Also disclosed is a composition for detecting an analyte comprising a specific binding molecule, a carrier, and a block group, wherein the block group comprises blocks, and wherein the blocks comprise reporter signals. The reporter signals can have a common property, wherein the common property can allow the reporter signals to be distinguished or separated from molecules lacking the common property, wherein the reporter signals can be altered, wherein the altered forms of each reporter signal can be distinguished from every other altered form of reporter signal.

The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. Alteration of the reporter signals also can alter their charge. The common property can be mass-to-charge ratio, wherein the reporter signals can be altered by altering their charge, wherein the altered forms of the labeled proteins can be distinguished via differences in the mass-to-charge ratio of the altered forms of reporter signals. The block group can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The block group can comprise ten or more different reporter signals.

The reporter signals can be peptides, oligonucleotides, carbohydrates, polymers, oligopeptides, or peptide nucleic acids. The reporter signals can be associated with, or coupled to, specific binding molecules, wherein each reporter signal can be associated with, or coupled to, a different specific binding molecule. The reporter signals can be associated with, or coupled to, decoding tags, wherein each reporter signal can be associated with, or coupled to, a different decoding tag. The reporter signals comprise peptides, wherein the peptides can have the same mass-to-charge ratio. The peptides can have the same amino acid composition. The peptides can have the same amino acid sequence. Each peptide can contain a different distribution of heavy isotopes. Each reporter signal peptide can contain a different distribution of substituent groups. Each peptide can have a different amino acid sequence. Each peptide can have a labile or scissile bond in a different location.

The reporter signals can be coupled to the proteins or peptides. The common property can allow the labeled proteins to be distinguished or separated from molecules lacking the common property. The common property need not be an affinity tag. One or more affinity tags can be associated with the reporter signals. The carrier can be selected from the group consisting of liposomes, microparticles, nanoparticles, and branched polymer structures. The carrier can be a liposome. The liposomes can be unilamellar vesicles. The vesicles can have an average diameter of 150 to 300 nanometers. The liposome can have an internal diameter of 200 nanometers.

The carrier can be a dendrimer. The dendrimer can be contacting a macromolecule selected from the group consisting of DNA, RNA, and PNA. The macromolecule can be an oligonucleotide between 20 and 300 nucleotides in length. The specific binding molecule can be selected from the group consisting of antibodies, ligands, binding proteins, receptor proteins, haptens, aptamers, carbohydrates, synthetic polyamides, and oligonucleotides. The specific binding molecule can be a binding protein. The binding protein can be a DNA binding protein. The DNA binding protein can contain a motif selected from the group consisting of a zinc finger motif, leucine zipper motif, and helix-turn-helix motif.

The specific binding molecule can be an oligonucleotide. The oligonucleotide can be between 10 and 40 nucleotides in length. The oligonucleotide can be between 16 and 25 nucleotides in length. The oligonucleotide can be a peptide nucleic acid. The oligonucleotide can form a triple helix with the target sequence. The oligonucleotide can comprise a psoralen derivative capable of covalently attaching the oligonucleotide to the target sequence. The specific binding molecule can be an antibody. The antibody can bind a protein. The blocks can be selected from the group consisting of oligonucleotides, carbohydrates, synthetic polyamides, peptide nucleic acids, antibodies, ligands, proteins, haptens, zinc fingers, aptamers, mass labels, and any combination of these.

The blocks can be peptide nucleic acids. The blocks can be capable of hybridizing specifically to a nucleic acid sequence. The length of the nucleic acid sequence can be between 10 and 35 nucleotides long. The length of the nucleic acid sequence can be between 15 and 20 nucleotides long. The blocks can be capable of being detected by a method selected from the group consisting of nuclear magnetic resonance, electron paramagnetic resonance, surface enhanced raman scattering, surface plasmon resonance, fluorescence, phosphorescence, chemiluminescence, resonance raman, microwave, mass spectrometry, mass spectrometry electrophoresis chromatography, and any combination of these.

The blocks can be capable of being detected through MALDI-TOF spectroscopy. The specific binding molecule and the carrier can be covalently linked. The carrier and the blocks can be covalently linked. The specific binding molecule and the carrier can be covalently linked. The specific binding molecule can comprise a first oligonucleotide and the carrier comprises a second oligonucleotide which can hybridize to the first oligonucleotide. The first oligonucleotide can be conjugated to an antibody which binds a protein. The blocks can be isobaric blocks.

Also disclosed is a set of detectors comprising a plurality of detectors, wherein each detector comprises a specific binding molecule, a carrier, and a block group, wherein the block group comprises blocks, wherein each block group has a different composition of blocks, and wherein the blocks comprise reporter signals. Each block group can have the same number of blocks. The block groups need not all have the same number of blocks. Each block group can have a different identity composition of blocks. The blocks can have the same amount composition. The blocks need not all have the same amount composition. Block groups that have the same identity composition of blocks can have different amount compositions of blocks. The reporter signals can have a common property, wherein the common property can allow the reporter signals to be distinguished or separated from molecules lacking the common property, wherein the reporter signals can be altered, wherein the altered forms of each reporter signal can be distinguished from every other altered form of reporter signal.

The common property is mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. Alteration of the reporter signals also can alter their charge. The common property can be mass-to-charge ratio, wherein the reporter signals can be altered by altering their charge, wherein the altered forms of the labeled proteins can be distinguished via differences in the mass-to-charge ratio of the altered forms of reporter signals.

The reporter signals that comprise the set of detectors can comprise a set of reporter signals, wherein the set of reporter signals can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The set of reporter signals can comprise ten or more different reporter signals. The reporter signals can be peptides, oligonucleotides, carbohydrates, polymers, oligopeptides, or peptide nucleic acids. The reporter signals can be associated with, or coupled to, specific binding molecules, wherein each reporter signal is associated with, or coupled to, a different specific binding molecule.

The reporter signals can be associated with, or coupled to, decoding tags, wherein each reporter signal can be associated with, or coupled to, a different decoding tag. The reporter signals can comprise peptides, wherein the peptides have the same mass-to-charge ratio. The peptides can have the same amino acid composition. The peptides can have the same amino acid sequence. Each peptide can contain a different distribution of heavy isotopes. Each reporter signal peptide can contain a different distribution of substituent groups. Each peptide can have a different amino acid sequence. Each peptide can have a labile or scissile bond in a different location.

The reporter signals can be coupled to the proteins or peptides. The common property can allow the labeled proteins to be distinguished or separated from molecules lacking the common property. The common property need not be an affinity tag. One or more affinity tags can be associated with the reporter signals.

Also disclosed is a set of block groups comprising a plurality of block groups, wherein each block group comprises blocks, wherein the blocks comprise reporter signals, wherein the reporter signals have a common property, wherein the common property allows the reporter signals to be distinguished or separated from molecules lacking the common property, wherein the reporter signals can be altered, wherein the altered forms of each reporter signal can be distinguished from every other altered form of reporter signal.

Also disclosed is a set of blocks comprising a plurality of blocks, wherein the blocks comprise reporter signals, wherein the reporter signals have a common property, wherein the common property allows the reporter signals to be distinguished or separated from molecules lacking the common property, wherein the reporter signals can be altered, wherein the altered forms of each reporter signal can be distinguished from every other altered form of reporter signal.

Also disclosed is a kit comprising a set of detectors, wherein the set of detectors comprises a plurality of detectors, wherein each detectors comprises a specific binding molecule, a carrier, and a block group, wherein the block group comprises blocks, and wherein the blocks comprise reporter signals. The reporter signals can have a common property, wherein the common property can allow the reporter signals to be distinguished or separated from molecules lacking the common property, wherein the reporter signals can be altered, wherein the altered forms of each reporter signal can be distinguished from every other altered form of reporter signal.

Also disclosed is a mixture comprising a set of detectors and a target sample, wherein the set of detectors comprises a plurality of detectors, wherein each detectors comprises a specific binding molecule, a carrier, and a block group, wherein the block group comprises blocks, and wherein the blocks comprise reporter signals. The reporter signals can have a common property, wherein the common property can allow the reporter signals to be distinguished or separated from molecules lacking the common property, wherein the reporter signals can be altered, wherein the altered forms of each reporter signal can be distinguished from every other altered form of reporter signal.

### Illustrations

A. Illustration 1

Combinatorially encoded bead analysis of a single protein sample using 4095 antibodies, one specific antibody per bead. Readout of bound proteins is direct. In this illustration, the detectors comprise beads with antibodies and mass tags attached. The beads are the carriers, the antibodies are the specific binding molecules, and the mass tags are the blocks making up the block groups. The mass tags are isobaric reporter signals. For convenience, in this illustration, the detectors are referred to as “beads.” However, these “beads” are beads with antibodies and mass tags attached.

1. Bead classes (that is, detector classes) are prepared in separate vessels, by covalent binding of a unique combination of isobaric mass tags (that is, isobaric blocks, or, more specifically, isobaric reporter signals), choosing the combinations by mixing members of 12 types of isobaric mass tags. The number of possible combinations of tags is 4095. Each class of coded bead (that is, each class of detector) is subsequently derivatized to obtain covalent binding of a specific antibody, and the process is repeated for a total of 4095 different antibodies. The combined bead/mass tag/antibody structure is a bead detector.

2. Anywhere from 2 to 4095 classes of bead detectors (100 beads of each class) are mixed together in a single reaction vessel, and contacted with a biological sample, where the biological sample comprises a cell lysate from a fine needle aspirate from the prostate.

3. The beads are washed, spread on a MALDI plate, avoiding aggregation or clumping, and coated with matrix. The plate is inserted in a mass spectrometer, where said mass spectrometer has the capability to direct laser shots at individual beads, either deterministically using video guidance, or stochastically using a raster matrix.

4. The MALDI analysis for tag decoding is performed in a tandem mass spectrometer, using Quadrupole settings for single-ion filtering, followed by a collision stage for ion fragmentation, and finally TOF spectrometry of the peptide fragments that arise from the original single-ion. In the second stage, signal to noise of the TOF measurement is much larger than in a conventional MS experiment. The unique combination of mass tags (that is, the unique combination of blocks) occurring on the surface of each class of bead is decoded from the MS/MS mass spectrum.

5. The spectrometer is then switched to single-dimension MS-TOF mode, and new series of laser shots is performed on the same bead, to collect the spectrum of the proteins bound by the antibodies on the surface of the single, previously decoded bead.

6. The mechanical stage is moved, and the MALDI process of steps 4 and 5 is repeated for a total of 30,000 beads, in order to sample beads of each of the different 4095 classes at least once, and preferably, multiple times.

B. Illustration 2

Combinatorially encoded bead analysis of a 32 tag-encoded protein sample using 4095 antibodies, one specific antibody per bead. In this illustration, the detectors comprise beads with antibodies and mass tags attached. The beads are the carriers, the antibodies are the specific binding molecules, and the mass tags are the blocks making up the block groups. The mass tags are isobaric reporter signals. For convenience, in this illustration, the detectors are referred to as “beads.” However, these “beads” are beads with antibodies and mass tags attached. This illustration combines multiple tag analysis with the disclosed method.

1. Bead classes (that is, detector classes) are prepared in separate vessels, by covalent binding of a unique combination of isobaric mass tags (that is, isobaric blocks, or, more specifically, isobaric reporter signals), choosing the combinations by mixing members of 12 types of isobaric mass tags. The number of possible combinations of the tags is 4095. Each class of coded bead is subsequently derivatized to obtain covalent binding of a specific antibody, and the process is repeated for a total of 4095 different antibodies.

2. The beads are mixed together in a single reaction vessel, and contacted with a complex biological sample, where the sample comprises a mixture of 32 previously prepared coded samples, where coding of each of the 32 samples is performed by covalent labeling, as described in PCT Application WO 00/68434, with one of 32 different reporter molecules comprising specific binding molecules and oligonucleotides.

3. After sample binding, the beads are washed and contacted with a solution of 32 PNA-peptide chimeric decoding tags, where the 32 PNA-peptide chimeras are isobaric with each other and are each capable of recognizing specifically a unique corresponding reporter molecule associated with the protein samples.

4. The beads are washed, spread on a MALDI plate, and coated with matrix. The plate is inserted in a mass spectrometer, where the mass spectrometer has the capability to direct laser shots at individual beads, either deterministically or stochastically.

5. The MALDI-TOF analysis for bead tag decoding (that is, detector decoding or block group decoding) is performed in a tandem mass spectrometer, using Quadrupole settings for single-ion filtering, followed by a collision stage for ion fragmentation, and finally TOF spectrometry of the peptide fragments that arise from the original single-ion. The unique combination of mass tags occurring on the surface of each class of bead is decoded from the MS/MS mass spectrum.

6. The single-ion filter of the Quadrupole instrument is then switched to the mass of the PNA-peptide chimeric decoding tags (which are isobaric), and new series of laser shots is performed on the same bead, to collect the signal spectrum of the 32 tagged proteins bound by the antibodies on the surface of a single bead. Thus, the tag decoding analysis generates a signal profile corresponding to all 32 pre-mixed biological samples, for those labeled proteins that bind to the unique antibody bound on a single bead. The decoding tags identify the sample involved and the bead encoding tags (that is, the blocks on the beads) identify the bead, and thus the protein, involved.

7. The mechanical stage is moved, and the MALDI process of steps 4 and 5 is repeated for a total of 30,000 beads, in order to sample beads of each of the different 4095 classes at least once, and preferably, multiple times.

8. The analysis generates a protein profile consisting of 32*4095=31,040 protein expression values for a single 32-sample experiment. However, since a total of 30,000 beads are analyzed, there is significant over-sampling (3- to 7-fold) for most of the antibodies (the total number of data points is 32*30,000=960,000).

C. Illustration 3

Combinatorially encoded bead analysis of a 32 tag-encoded protein sample using 4095 antibodies, one specific antibody per bead. In this illustration, the detectors comprise beads with antibodies and mass tags attached. The beads are the carriers, the antibodies are the specific binding molecules, and the mass tags are the blocks making up the block groups. The mass tags are isobaric reporter signals. For convenience, in this illustration, the detectors are referred to as “beads.” However, these “beads” are beads with antibodies and mass tags attached. This illustration is similar to illustration 2, but in this illustration, the decoding tags and the bead encoding tags (that is, the mass tags) belong to the same isobaric set (that is, the decoding tags and the mass tags are all isobaric).

1. Bead classes (that is, detector classes) are prepared in separate vessels, by covalent binding of a unique combination of isobaric mass tags (that is, isobaric blocks, or, more specifically, isobaric reporter signals), choosing the combinations by mixing members of 12 types of PNA-peptide isobaric mass tags. The number of possible combinations of the tags is 4095. Each class of coded bead is subsequently derivatized to obtain covalent binding of a specific antibody, and the process is repeated for a total of 4095 different antibodies.

2. The beads are mixed together in a single reaction vessel, and contacted with a complex biological sample, where the sample comprises a mixture of 32 previously prepared coded samples, where coding of each of the 32 samples is performed by covalent labeling, as described in PCT Application WO 00/68434, with one of 32 different reporter molecules comprising specific binding molecules and oligonucleotides.

3. After sample binding, the beads are washed and contacted with a solution of 32 PNA-peptide chimeric decoding tags, where the 32 PNA-peptide chimeras are isobaric with each other and are each capable of recognizing specifically a unique corresponding reporter molecule associated with the protein samples. Furthermore, the 32 PNA-peptide chimeras are also members of the same isobaric set as (that is, they are isobaric to) the 12 mass tags encoding the beads, for a total of 44 isobaric components.

4. The beads are washed, spread on a MALDI plate, and coated with matrix. The plate is inserted in a mass spectrometer, where said mass spectrometer has the capability to direct laser shots at individual beads, either deterministically or stochastically.

5. The MALDI-TOF analysis for bead tag decoding (that is, detector decoding or block group decoding) is performed in a tandem mass spectrometer, using Quadrupole settings for single-ion filtering, followed by a collision stage for ion fragmentation, and finally TOF spectrometry of the PNA-peptide fragments that arise from the original single-ion. The unique combination of mass tags occurring on the surface of each class of bead is decoded from the MS/MS mass spectrum. The same Quadrupole instrument readout provides identification of the PNA-peptide chimeric decoding tags. One thus obtains, as part of the same readout, the signal spectrum of the 32 tagged proteins (from the 32 samples) bound by the antibody on the surface of a single bead. Thus, the multiple tag decoding analysis generates the multiple tag analysis signal profile corresponding to all 32 pre-mixed biological samples, for those labeled proteins that bind to the unique antibody bound on a single bead. The decoding tags identify the sample involved and the bead encoding tags (that is, the blocks on the beads) identify the bead, and thus the protein, involved.

6. The mechanical stage is moved, and the MALDI process of steps 4 and 5 is repeated for a total of 30,000 beads, in order to sample beads of each of the different 4095 classes at least once, and preferably, multiple times.

The analysis generates a protein profile consisting of 32*4095=131,040 protein expression values for a single 32-sample experiment. However, since a total of 30,000 beads are analyzed, there is significant over-sampling (3- to 7-fold) for most of the antibodies (the total number of data points is 32*30,000=960,000).

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a “, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

The present invention may be understood more readily by reference to the foregoing detailed description of preferred embodiments of the invention and the Illustrations included therein and to the Figures and their previous and following description. It is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. “Optional” or “optionally” means that the subsequently described event or circumstance may or may not occur, and that the description includes instances where said event or circumstance occurs and instances where it does not.

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular detector, carrier, block group, or block is disclosed and discussed and a number of modifications that can be made to a number of molecules including the detector, carrier, block group, or block are discussed, specifically contemplated is each and every combination and permutation of detector, carrier, block group, or block and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

I claim:

1. A method of detecting analytes, the method comprising
associating one or more detectors with one or more target samples, wherein the detectors each comprise a specific binding molecule, a carrier, and a block group, wherein the block group comprises blocks, and detecting the block group.

2. The method of claim 1, wherein the blocks have the same amount composition.

3. The method of claim 1, wherein the blocks do not all have the same amount composition.

4. The method of claim 1, wherein a plurality of detectors are associated with the one or more target samples, wherein the block group of each detector has a different composition of blocks.

5. The method of claim 4, wherein each block group has the same number of blocks.

6. The method of claim 4, wherein the block groups do not all have the same number of blocks.

7. The method of claim 4, wherein each block group has a different identity composition of blocks.

8. The method of claim 4, wherein the blocks have the same amount composition.

9. The method of claim 4, wherein the blocks do not all have the same amount composition.

10. The method of claim 4, wherein block groups that have the same identity composition of blocks have different amount compositions of blocks.

11. The method of claim 1, wherein the blocks are peptide nucleic acids.

12. The method of claim 1, wherein the blocks are capable of hybridizing specifically to an oligonucleotide reporter tag.

13. The method of claim 12, wherein the length of the oligonucleotide reporter tag is between 10 and 35 nucleotides long.

14. The method of claim 12, wherein the length of the oligonucleotide reporter tag is between 15 and 20 nucleotides long.

15. The method of claim 1, wherein the blocks are capable of being detected by a method selected from the group consisting of nuclear magnetic resonance, electron paramagnetic resonance, surface enhanced raman scattering, surface plasmon resonance, fluorescence, phosphorescence, chemiluminescence, resonance raman, microwave, mass spectrometry, mass spectrometry electrophoresis chromatography, and any combination of these.

16. The method of claim 1, wherein the blocks are capable of being detected through MALDI-TOF spectroscopy.

17. The method of claim 1, wherein the blocks are isobaric blocks.

18. The method of claim 17, wherein a plurality of detectors are associated with one or more target samples, wherein the blocks of each detector are different.

19. The method of claim 18, wherein all of the blocks of all of the detectors have the same mass-to-charge ratio.

20. The method of claim 19, wherein the blocks are altered by altering their mass, charge, or both, wherein the altered forms of the blocks are distinguished via differences in the mass-to-charge ratio of the altered forms of the blocks.

21. The method of claim 1, wherein the carrier is selected from the group consisting of beads, liposomes, microparticles, nanoparticles, and branched polymer structures.

22. The method of claim 1, wherein the carrier is a bead.

23. The method of claim 1, wherein the carrier is a liposome or microbead.

24. The method of claim 23, wherein the liposomes are unilamellar vesicles.

25. The method of claim 24, wherein the vesicles have an average diameter of 150 to 300 nanometers.

26. The method of claim 21, wherein the liposome has an internal diameter of 200 nanometers.

27. The method of claim 1, wherein the carrier is a dendrimer.

28. The method of claim 27, wherein the dendrimer is contacting a macromolecule selected from the group consisting of DNA, RNA, and PNA.

29. The method of claim 28, wherein the macromolecule is an oligonucleotide between 20 and 300 nucleotides in length.

30. The method of claim 1, wherein the specific binding molecule is selected from the group consisting of antibodies, ligands, binding proteins, receptor proteins, haptens, aptamers, carbohydrates, synthetic polyamides, and oligonucleotides.

31. The method of claim 1, wherein the specific binding molecule is a binding protein.

32. The method of claim 31, wherein the binding protein is a DNA binding protein.

33. The method of claim 31, wherein the DNA binding protein contains a motif selected from the group consisting of a zinc finger motif, leucine zipper motif, and helix-turn-helix motif.

34. The method of claim 33, wherein the specific binding molecule is an oligonucleotide.

35. The method of claim 33, wherein the oligonucleotide is between 10 and 40 nucleotides in length.

36. The method of claim 33, wherein the oligonucleotide is between 16 and 25 nucleotides in length.

37. The method of claim 33, wherein the oligonucleotide is a peptide nucleic acid.

38. The method of claim 33, wherein the oligonucleotide forms a triple helix with the target sequence.

39. The method of claim 33, wherein the oligonucleotide comprises a psoralen derivative capable of covalently attaching the oligonucleotide to the target sequence.

40. The method of claim 1, wherein the specific binding molecule is an antibody.

41. The method of claim 40, wherein the antibody binds a protein.

42. The method of claim 1, wherein the blocks are oligonucleotides, carbohydrates, synthetic polyamides, peptide nucleic acids, antibodies, ligands, proteins, haptens, zinc fingers, aptamers, mass labels, or any combination of these.

43. The method of claim 1, wherein the specific binding molecule and the carrier are covalently linked.

44. The method of claim 1, wherein the carrier and the blocks are covalently linked.

45. The method of claim 44, wherein the specific binding molecule and the carrier are covalently linked.

46. The method of claim 1, wherein the specific binding molecule comprises a first oligonucleotide and the carrier comprises a second oligonucleotide which can hybridize to the first oligonucleotide.

47. The method of claim 46, wherein the first oligonucleotide is conjugated to an antibody which binds a protein.

48. A composition for detecting an analyte comprising a specific binding molecule, a carrier, and a block group.

49. The composition of claim 48, wherein the carrier is selected from the group consisting of liposomes, microparticles, nanoparticles, and branched polymer strictures.

50. The composition of claim 48, wherein the carrier is a liposome.

51. The composition of claim 50, wherein the liposomes are unilamellar vesicles.

52. The composition of claim 51, wherein the vesicles have an average diameter of 150 to 300 nanometers.

53. The composition of claim 50, wherein the liposome has an internal diameter of 200 nanometers.

54. The composition of claim 48, wherein the carrier is a dendrimer.

55. The composition of claim 54, wherein the dendrimer is contacting a macromolecule selected from the group consisting of DNA, RNA, and PNA.

56. The composition of claim 55, wherein the macromolecule is an oligonucleotide between 20 and 300 nucleotides in length.

57. The composition of claim 48, wherein the specific binding molecule is selected from the group consisting of antibodies, ligands, binding proteins, receptor proteins, haptens, aptamers, carbohydrates, synthetic polyamides, and oligonucleotides.

58. The composition of claim 48, wherein the specific binding molecule is a binding protein.

59. The composition of claim 58, wherein the binding protein is a DNA binding protein.

60. The composition of claim 58, wherein the DNA binding protein contains a motif selected from the group consisting of a zinc finger motif, leucine zipper motif, and helix-turn-helix motif.

61. The composition of claim 48, wherein the specific binding molecule is an oligonucleotide.

62. The composition of claim 61, wherein the oligonucleotide is between 10 and 40 nucleotides in length.

63. The composition of claim 61, wherein the oligonucleotide is between 16 and 25 nucleotides in length.

64. The composition of claim 61, wherein the oligonucleotide is a peptide nucleic acid.

65. The composition of claim 61, wherein the oligonucleotide forms a triple helix with the target sequence.

66. The composition of claim 65, wherein the oligonucleotide comprises a psoralen derivative capable of covalently attaching the oligonucleotide to the target sequence.

67. The composition of claim 48, wherein the specific binding molecule is an antibody.

68. The composition of claim 67, wherein the antibody binds a protein.

69. The composition of claim 48, wherein the blocks are selected from the group consisting of oligonucleotides, carbohydrates, synthetic polyamides, peptide nucleic acids, antibodies, ligands, proteins, haptens, zinc fingers, aptamers, mass labels, and any combination of these.

70. The composition of claim 48, wherein the blocks are peptide nucleic acids.

71. The composition of claim 48, wherein the blocks are capable of hybridizing specifically to an oligonucleotide reporter tag.

72. The composition of claim 71, wherein the length of the oligonucleotide reporter tag is between 10 and 35 nucleotides long.

73. The composition of claim 71, wherein the length of the oligonucleotide reporter tag is between 15 and 20 nucleotides long.

74. The composition of claim 48, wherein the blocks are capable of being detected by a method selected from the group consisting of nuclear magnetic resonance, electron paramagnetic resonance, surface enhanced raman scattering, surface plasmon resonance, fluorescence, phosphorescence, chemiluminescence, resonance raman, microwave, mass spectrometry, mass spectrometry electrophoresis chromatography, and any combination of these.

75. The composition of claim 48, wherein the blocks are capable of being detected through MALDI-TOF spectroscopy.

76. The composition of claim 48, wherein the specific binding molecule and the carrier are covalently linked.

77. The composition of claim 48, wherein the carrier and the blocks are covalently linked.

78. The composition of claim 77, wherein the specific binding molecule and the carrier are covalently linked.

79. The composition of claim 48, wherein the specific binding molecule comprises a first oligonucleotide and the carrier comprises a second oligonucleotide which can hybridize to the first oligonucleotide.

80. The composition of claim 79, wherein the first oligonucleotide is conjugated to an antibody which binds a protein.

81. The composition of claim 48, wherein the blocks are isobaric blocks.

82. A set of detectors comprising a plurality of detectors,
wherein each detectors comprises a specific binding molecule, a carrier, and a block group, wherein the block group comprises blocks, wherein each block group has a different composition of blocks.

83. The set of claim 82, wherein each block group has the same number of blocks.

84. The set of claim 82, wherein the block groups do not all have the same number of blocks.

85. The set of claim 82, wherein each block group has a different identity composition of blocks.

86. The set of claim 82, wherein the blocks have the same amount composition.

87. The set of claim 82, wherein the blocks do not all have the same amount composition.

88. The set of claim 82, wherein block groups that have the same identity composition of blocks have different amount compositions of blocks.

89. The set of claim 82, wherein the blocks comprise reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

90. The set of claim 89, wherein the common property is mass-to-charge ratio, wherein the reporter signals are altered by altering their mass, wherein the altered forms of the reporter signals can be distinguished via differences in the mass-to-charge ratio of the altered forms of reporter signals.

91. The set of claim 90, wherein the mass of the reporter signals is altered by fragmentation.

92. The set of claim 90, wherein alteration of the reporter signals also alters their charge.

93. The set of claim 89, wherein the common property is mass-to-charge ratio, wherein the reporter signals are altered by altering their charge, wherein the altered forms of the labeled proteins can be distinguished via differences in the mass-to-charge ratio of the altered forms of reporter signals.

94. The set of claim 89, wherein the set comprises two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals.

95. The set of claim 94, wherein the set comprises ten or more different reporter signals.

96. The set of claim 89, wherein the reporter signals are peptides, oligonucleotides, carbohydrates, polymers, oligopeptides, or peptide nucleic acids.

97. The set of claim 89, wherein the reporter signals are associated with, or coupled to, specific binding molecules, wherein each reporter signal is associated with, or coupled to, a different specific binding molecule.

98. The set of claim 89, wherein the reporter signals are associated with, or coupled to, decoding tags, wherein each reporter signal is associated with, or coupled to, a different decoding tag.

99. The set of claim 89, wherein the reporter signals comprise peptides, wherein the peptides have the same mass-to-charge ratio.

100. The set of claim 99, wherein the peptides have the same amino acid composition.

101. The set of claim 100, wherein the peptides have the same amino acid sequence.

102. The set of claim 101, wherein each peptide contains a different distribution of heavy isotopes.

103. The set of claim 101, wherein each reporter signal peptide contains a different distribution of substituent groups.

104. The set of claim 100, wherein each peptide has a different amino acid sequence.

105. The set of claim 100, wherein each peptide has a labile or scissile bond in a different location.

106. The set of claim 89, wherein the reporter signals are coupled to the proteins or peptides.

107. The set of claim 89, wherein the common property allows the labeled proteins to be distinguished or separated from molecules lacking the common property.

108. The set of claim 89, wherein the common property is not an affinity tag.

109. The set of claim 108, wherein one or more affinity tags are associated with the reporter signals.

110. A set of block groups comprising a plurality of block groups,
wherein each block group comprises blocks, wherein the blocks comprise reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

111. A set of blocks comprising a plurality of blocks,
wherein the blocks comprise reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

112. A kit comprising a set of detectors, wherein the set of detectors comprises a plurality of detectors,
wherein each detectors comprises a specific binding molecule, a carrier, and a block group, wherein the block group comprises blocks.

113. The kit of claim 112 wherein the blocks comprise reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

114. A mixture comprising
a set of detectors and a target sample,
wherein the set of detectors comprises a plurality of detectors, wherein each detectors comprises a specific binding molecule, a carrier, and a block group, wherein the block group comprises blocks.

115. The mixture of claim 114 wherein the blocks comprise reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

116. A method of detecting analytes, the method comprising
associating one or more detectors with one or more target samples, wherein the detectors each comprise a specific binding molecule, a carrier, and a block group, and
detecting the block group,
wherein the block group comprises blocks, wherein the blocks comprise reporter signals, wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal,
wherein the common property is mass-to-charge ratio, wherein the reporter signals are altered by altering their mass, wherein the altered forms of the reporter signals can be distinguished via differences in the mass-to-charge ratio of the altered forms of reporter signals,
wherein the mass of the reporter signals is altered by fragmentation,
wherein the block group comprises ten or more different reporter signals,
wherein the reporter signals comprise peptides, wherein the peptides have the same mass-to-charge ratio, wherein the peptides have the same amino acid composition, wherein the peptides have the same amino acid sequence, wherein each peptide contains a different distribution of heavy isotopes.
